(19) United States
(12) Patent Application Publication
(10) Pub. No.: US 2017/0260275 A1
(43) Pub. Date: Sep. 14, 2017

(54) MYOSTATIN OR ACTIVIN ANTAGONISTS FOR THE TREATMENT OF SARCOPENIA

(71) Applicant: Novartis AG, Basel (CH)

(72) Inventors: Patrick KORTEBEIN, El Dorado Hills, CA (US); Daniel ROOKS, Cambridge, MA (US); Lloyd B. KLICKSTEIN, Newton, MA (US); Ronenn ROUBENOFF, Basel (CH); David GLASS, Cambridge, MA (US); Estelle TRIFILIEFF, Dietwiller (FR); Dimitris PAPANICOLAOU, Tenafty, NJ (US)

(21) Appl. No.: 15/529,594

(22) PCT Filed: Dec. 4, 2015
(86) PCT No.: PCT/IB2015/059369

§ 371 Date: May 25, 2017

Related U.S. Application Data
(60) Provisional application No. 62/088,802, filed on Dec. 8, 2014.

(51) Int. Cl. C07K 16/28

(52) U.S. Cl. CPC C07K 16/2863; C07K 2317/76; A61K 2039/545

(57) ABSTRACT

The present invention relates to myostatin or activin antagonists, dose regimen and pharmaceutical compositions thereof, for the treatment of sarcopenia, in particular age-related sarcopenia. Especially, the myostatin or activin antagonist bimagrumab was found to be beneficial in the treatment of older adults with sarcopenia with respect to increasing their skeletal muscle strength and function.

### FIELD OF THE INVENTION

The present invention relates to myostatin, activin or GDF11 antagonists, dose regimen and pharmaceutical compositions thereof, for use in the treatment of sarcopenia, in particular age-related sarcopenia.

### BACKGROUND OF THE INVENTION

Sarcopenia, the age-associated loss of skeletal muscle mass and physical function (Cruz-Jentoft et al 2010; Fielding et al 2011), affects approximately 30% of American men and women over the age of 60 and 50% older than 80 years (Baumgartner et al 1998). Sarcopenia is thought to result in mobility disability in 2-5% of elderly adults (Dam et al 2014). Loss of skeletal muscle mass and strength are common consequences of many chronic diseases, hospitalizations and normal aging and are strongly associated with morbidity, disability, mortality and loss of independence (Janssen et al 2004). A decline in muscle mass and strength in the elderly often manifests as reduced physical functional capacity leading to lower quality of life and an increased risk of adverse health events (e.g., falls and fractures subsequent to falls). Currently, there is no standard treatment for the loss of skeletal muscle mass and function seen with aging.

Multiple consensus groups have proposed definitions for sarcopenia based upon changes in both muscle mass and function. Thus, diagnosis depends on documentation of low muscle mass plus the presence of low muscle function (low muscle strength/weakness or low physical performance) (Cruz-Jentoft et al 2010; Muscaritoli et al 2010; Fielding et al 2011, Studenski et al 2014).

The European Working Group on Sarcopenia in Older People (EWGSOP) recommended thresholds for defining sarcopenia that were based on the mean muscle mass in a normative healthy young adult population, with cutoff points calculated as two standard deviations below the mean reference value. This threshold is operationalized using an appendicular skeletal muscle index (ASMI, skeletal muscle of the upper and lower limbs in kg/height in m2) by dual energy X-ray absorptiometry (DXA) of ≦7.26 kg/m² for men and ≦5.5 kg/m² for women.

A similar consensus definition on sarcopenia was recently published from four other collaborative special interest groups—“Cachexia-anorexia in chronic wasting diseases”, “International Working Group on Sarcopenia”, “Nutrition in Geriatrics” and the “Asian Working Group for Sarcopenia”—that recommended low muscle mass as well as usual gait speed as the preferred parameter of physical function (Muscaritoli et al 2010; Fielding et al 2011; Chen et al 2014).

Frailty is another prevalent geriatric syndrome with a well characterized, relatively discrete phenotype that also results in a number of adverse sequelae including falls, hospitalization, institutionalization and death (Fried et al 2001). It is generally acknowledged that the pathophysiologic process of sarcopenia underlies the functional deficits of frail individuals (Cruz-Jentoft et al 2010). Due to the recognized overlap of these geriatric conditions, in 2013 the European Union Innovative Medicines Initiative (IMI) initiated a call for proposals to develop diagnostic criteria and treatment initiatives for ‘physical frailty and sarcopenia’ (PF&S). The IMI PF&S consortium aims to establish a consensus definition of PF&S that will be presented at the 2014 meeting of the European Geriatrics Society in Rotterdam, subsequently published and applied to a large (n=1500) EU clinical trial on the effect of exercise on PF&S. PF&S is, however, not widely used in the medical or scientific community, nor is there consensus on its definition at this stage. In terms of the present proposal, it is important to note that the definition of PF&S is based on the EWGSOP definition of sarcopenia, which is also the definition Novartis proposes for the bimagrumab program. Thus, the population for the phase IIb sarcopenia clinical trial is expected to be similar, if not identical, to the PF&S population. Although this is a rapidly emerging field with the possibility of additional changes in definition over the corning years, the community has been converging on the current definitions for the past decade, and large-scale changes seem unlikely. If they occur prior to initiating a phase III trial(s) in sarcopenia, this would be would taken under consideration, in consultation with health authorities.

Easy to perform in both clinical and research environments, gait speed is a common component of comprehensive geriatric assessment and care in many countries. In addition, there is a substantial body of epidemiologic and intervention based literature demonstrating a strong association between slowed and declining gait speed and future adverse physical status and health outcomes, including mortality (Studenski et al 2011). The two gait speed cutoff points recommended in the consensus statements for the diagnosis of sarcopenia are <0.8 m/s and 1 m/s in the 4 m walking test to include patients at increased risk of functional decline (Cruz-Jentoft et al 2010; Fielding et al 2011). The largest analysis to date, of 26,000 patients in observational data from multiple studies, further supports the 0.8 m/s cutoff to define the population at increased risk for adverse health events (Dam et al 2014).

Based on its “Sarcopenia Project” the Foundation for the National Institute of Health (FNIH) set the focus in diagnosis of sarcopenia on weakness and low muscle mass (Studenski et al 2014). FNIH recommended as cutoff points for weakness <26 kg for men and <16 kg for women in the grip strength test. Recommended cutoff points for low muscle mass were set to <0.789 for men and <0.512 for women referring to the appendicular lean body mass adjusted for BMI.

An accelerated loss of muscle mass, strength and physical function in the large and rapidly growing global aging population represents a substantial, unmet medical need. Therefore, pharmacotherapeutics that can promote skeletal muscle hypertrophy and improve patient muscle function high are highly desired.

### SUMMARY OF THE INVENTION

Muscle Regulation and the ActRII Receptors

Several members of the transforming growth factor beta (TGF-β) superfamily, including myostatin, activin A, and growth differentiation factor 11 (GDF11), negatively regulate skeletal muscle mass in animals and humans throughout the lifecycle. Ligand signaling occurs via type II activin receptors (both ActRIIA and B; and primarily the Smad 2/3 pathway), to inhibit muscle protein synthesis and myocyte differentiation and proliferation. The absence of any of these ligands in developing animals and humans results in a hypermuscular phenotype with an increased number and size of muscle fibers. A postpartum reduction of myostatin levels results in the hypertrophy of skeletal muscle due to an increase in the size of existing myofibers (Lee et al 2005; Lee et al 2010; Trendelenburg et al 2012). Thus, the capacity for modulating muscle growth by perturbing this signaling pathway at the receptor level is much more substantial than previously appreciated by direct anti-myostatin approaches.

Bimagrumab

Bimagrumab, the pharmaceutically active compound used in accordance with the present invention, is a fully human, monoclonal antibody (modified IgG1, 234-235-Ala-Ala, A2) developed to bind competitively to activin receptor type II (ActRII) with greater affinity than its natural ligands that limit muscle mass growth, including myostatin and activin. Bimagrumab is cross-reactive with human and mouse ActRIIA and ActRIIB and effective on human, cynomolgus, mouse and rat skeletal muscle cells. Bimagrumab binds with extremely high affinity (KD 1.7±0.3 pM) to human ActRIIB and with relatively lower affinity to human ActRIIA (KD 434±25 pM), and is formulated for intravenous (i.v.) administration.

The present invention is based on the therapeutic approach that sufficiently blocking myostatin or activin binding to their receptors ActRII (preferably ActRIIB and ActRIIA, or ActRIIA or ActRIIB either alone) will significantly reduce the activity of myostatin and other ligands that inhibit skeletal muscle growth acting at the receptors, while allowing some of those ligands to perform other physiologic functions via alternative type II receptors (Upton et al 2009). Other approaches to reducing myostatin activity, i.e. competitive soluble ActRII, creating a soluble receptor sink may deplete a range of ActRII ligands with activities at other receptors, potentially creating a greater safety risk than using a receptor antagonist antibody like bimagrumab.

Other approaches include the use of or antibodies binding myostatin such as LY2495655 (Eli Lilly), which will then inhibit or reduce signalling through the ActRII receptors.

As a potent inhibitor of ActRII, bimagrumab blocks the effects of myostatin, activin A, GDF11, and possibly other ligands working through those receptors.

The present invention therefore provides a myostatin or activin antagonist, preferably a myostatin binding molecule or antibody, and more preferably an anti-ActRII receptor antibody, most preferably bimagrumab, for use in the treatment of human patients suffering from age-related sarcopenia.

In a similar aspect the present invention provides a myostatin antagonist, preferably a myostatin binding molecule or antibody, and more preferably an anti-ActRII receptor antibody, most preferably bimagrumab, for use in the treatment of human patients suffering from frailty or physical frailty or physical frailty & sarcopenia.

It has been observed that Activin A levels might be increasing with age (unpublished data). Activin can be any of activin A or activin B or a dimer thereof, Activin AB.

Thus, a further approach includes the use of an activin antagonist which will inhibit or reduce signalling through the ActRII receptors.

It is also known that activin when overexpressed is reversibly inducing muscle wasting. These findings highlight the therapeutic potential of targeting activins in addition to myostatin in cachexia and potentially in other muscle wasting disorders including sarcopenia (Chen et al., FASEB J. 2014 April; 28(4):1711-23).

In a similar aspect the present invention provides an activin antagonist, preferably an anti-ActRII receptor antibody, most preferably bimagrumab, for use in the treatment of human patients suffering from frailty or physical frailty or physical frailty & sarcopenia.

The present invention further provides a specific dose regimen for the myostatin or activin antagonist bimagrumab for use in the treatment of human patients suffering from age-related sarcopenia. According to the present invention bimagrumab is administered intravenously at a dose regimen of about 70 mg, about 210 mg, or about 700 mg, once every 4 weeks. The term “about” means herein ±20%.

The advantage of said treatment is that the patients improve with respect to their physical performance, their muscle strength and/or their muscle mass/volume.

### BRIEF DESCRIPTION OF THE DRAWINGS

In the following the present invention is described in detail with reference to accompanying figures in which:

FIG. 1 shows the arithmetic mean (SD) concentrations of bimagrumab for the cohorts 1, 2 and 3.

Cohort 1: Subjects were given 3 monthly i.v. infusions of 10 mg/kg (+)

Cohort 2: Subjects were given 3 monthly i.v. infusions of 3 mg/kg (o)

Cohort 3: Subjects were given a single i.v. infusion of 30 mg/kg (x)

### DETAILED DESCRIPTION OF THE INVENTION

Herein after, the present invention is described in further detail and is exemplified.

The present invention is provided in its following aspects:
1. A myostatin or activin antagonist for use in the treatment of human patients suffering from age-related sarcopenia.
2. A myostatin or activin antagonist for use according to aspects 1 wherein the human patients are men or post-menopausal women aged 50 years or older, preferably 60 years or older, more preferably 65 years or older, even more preferably 70 years or older.
   In one aspect, the invention relates to a myostatin or activin antagonist for use according to aspect 1 or 2 wherein the myostatin or activin antagonist is an anti-ActRII receptor inhibitor.
3. A myostatin or activin antagonist for use according to aspect 1 or 2 or to the previous aspect wherein the myostatin or activin antagonist is an anti-ActRII receptor antibody.
4. A myostatin or activin antagonist for use according to aspect 3 wherein the anti-ActRII receptor antibody is bimagrumab.
5. A myostatin or activin antagonist for use according to aspect 4 wherein the myostatin or activin antagonist is administered intravenously at a dose regimen of about 70 mg, about 210 mg, or about 700-750 mg, once every 4 weeks, wherein “about” has the meaning of ±20%.
6. A myostatin or activin antagonist for use according to any one of the aspects 1 to 5 wherein the treatment comprises an increase in physical performance (or mobility increase) latest after 24 weeks under treatment indicated by at least one of the following:
   (a) an increase of walking distance by at least 20 m, preferably at least 50 m in the 6 minute walk test (6MWT);
   (b) an decrease in time required to walk 400 m (400 m walk test) by at least 20 seconds, preferably 50 seconds;
   (c) an increase of short physical performance battery (SPPB) score by at least 0.3 points, preferably at least 0.5 points, more preferably at least 0.8 points, even more preferably at least 1.0 points;
   (d) an increase of gait speed over a 4-m course (4MGS) by at least 0.03 m/s, preferably at least 0.05 m/s, more preferably at least 0.08 m/s, even more preferably at least 0.10 m/s;
   compared to the data before treatment (baseline).
7. A myostatin or activin antagonist for use according to any one of the aspects 1 to 5 wherein the treatment comprises an increase in (skeletal) muscle mass indicated by at least one of the following:
   (a) an increase of appendicular skeletal muscle index (ASMI) to reach latest after 24 weeks under treatment a value of at least 7.26 kg/m² for men or at least 5.5 kg/m² for women, said ASMI being defined as appendicular skeletal muscle mass divided by the square of height;
   (b) an increase of appendicular lean (body) mass (AL(B)M) to reach latest after 24 weeks under treatment a value of at least 19.75 kg for men or at least 15.02 kg for women;
   (c) an increase of AL(B)M adjusted for body mass index (BMI) to reach latest after 24 weeks under treatment a value of at least 0.789 kg for men or at least 0.512 kg for women;
   (d) an increase of thigh muscle volume (TMV) of at least 5%, more preferably 7%, after 8 weeks under treatment, compared to the data before treatment (baseline);
   said ASMI and AL(B)M being measured by dual energy X-ray absorptiometry (DXA) and said TMV being measured by magnetic resonance imaging (MRI).
8. A myostatin or activin antagonist for use according to any one of the aspects 1 to 5 wherein the treatment comprises an increase in muscle strength indicated by reaching a value of at least 26 kg, preferably 30 kg, for men or 16 kg, preferably 20 kg, for women in the handgrip strength test latest after 24 weeks under treatment.
9. A myostatin or activin antagonist for use according to any one of the aspects 1 to 5 wherein the treatment comprises an increase in physical performance (or mobility increase) indicated by at least one of the criteria of aspect 6 and an increase in (skeletal) muscle mass indicated by at least one of the criteria of aspect 7 latest after 24 weeks under treatment.
10. A myostatin or activin antagonist for use according to any one of the aspects 1 to 5 wherein the treatment comprises an increase in physical performance (or mobility increase) indicated by at least one of the criteria of aspect 6 and an increase in muscle strength indicated by the criteria of aspect 8 latest after 24 weeks under treatment.
11. A myostatin or activin antagonist for use according to any one of the aspects 1 to 5 wherein the treatment comprises an increase in skeletal muscle mass indicated by at least one of the criteria of aspect 7 and an increase in muscle strength indicated by the criteria of aspect 8 latest after 24 weeks under treatment.
12. A myostatin or activin antagonist for use according to any one of the aspects 1 to 5 wherein the treatment comprises an increase in physical performance (or mobility increase) indicated by at least one of the criteria of aspect 6 and an increase in skeletal muscle mass indicated by at least one of the criteria of aspect 7 and an increase in muscle strength indicated by the criteria of aspect 8 latest after 24 weeks under treatment.

In a preferred embodiment, the present invention provides the myostatin antagonist bimagrumab for use according to any one of the aspects 1 to 5 wherein the treatment comprises an increase in skeletal muscle mass indicated by an increase of AL(B)M adjusted for body mass index (BMI) to reach latest after 24 weeks under treatment a value of at least 0.789 kg for men or at least 0.512 kg for women, said AL(B)M being measured by dual energy X-ray absorptiometry (DXA), and an increase in muscle strength indicated by reaching a value of at least 26 kg for men or 16 kg for women in the handgrip strength test latest after 24 weeks under treatment.

In another preferred embodiment, the present invention provides the myostatin antagonist bimagrumab for use according to any one of the aspects 1 to 5 wherein the treatment comprises an increase in skeletal muscle mass indicated by an increase of appendicular skeletal muscle index (ASMI) to reach latest after 24 weeks under treatment a value of at least 7.26 kg/m² for men or at least 5.5 kg/m² for women, said ASMI being defined as appendicular skeletal muscle mass divided by the square of height ASMI and being measured by dual energy X-ray absorptiometry (DXA), and an increase in muscle strength indicated by reaching a value of at least 30 kg for men or 20 kg for women in the handgrip strength test latest after 24 weeks under treatment.

In another preferred embodiment, the present invention provides the myostatin or activin antagonist bimagrumab for use according to any one of the aspects 1 to 5 wherein the treatment comprises an increase in physical performance (or mobility increase) indicated by an increase of gait speed over a 4-m course (4MGS) by at least 0.05 m/s compared to the data before treatment (baseline) and an increase in (skeletal) muscle mass indicated by an increase of appendicular skeletal muscle index (ASMI) to reach latest after 24 weeks under treatment a value of at least 7.26 kg/m² for men or at least 5.5 kg/m² for women, said ASMI being defined as appendicular skeletal muscle mass divided by the square of height and being measured by dual energy X-ray absorptiometry (DXA).
13. A myostatin or activin antagonist for use according to any one of the aspects 1 to 12 wherein sarcopenia is defined by the criterion of low physical performance (or mobility limitations) indicated by at least one of the following:
   (a) a walking distance of <400 m in the 6 minute walk test (6MWT);
   (b) a time of >15 min in the 400 m walk test;
   (c) a short physical performance battery (SPPB) score of ≦8;
   (d) a gait speed over a 4-m course of ≦1 m/s, preferably, ≦0.8 m/s, more preferably <0.8 m/s, even more preferably <0.8 m/s but 0.3 m/s.
14. A myostatin or activin antagonist for use according to any one of the aspects 1 to 12 wherein sarcopenia is defined by the criterion of low muscle mass (or low skeletal muscle mass) indicated by at least one of the following:
   (a) a appendicular skeletal muscle index (ASMI) of ≦7.26 kg/m² for men or ≦5.5 kg/m² for women, said ASMI being defined as appendicular skeletal muscle mass divided by the square of height;
   (b) an appendicular lean (body) mass (AL(B)M) of 19.75 kg for men or 15.02 kg for women;
   (c) an AL(B)M adjusted for body mass index (BMI) of 0.789 kg for men or ≦0.512 kg for women;
   said ASMI and AL(B)M being measured by dual energy X-ray absorptiometry (DXA) and said TMV being measured by magnetic resonance imaging (MRI).
15. A myostatin or activin antagonist for use according to any one of the aspects 1 to 12 wherein sarcopenia is defined by the criterion of low muscle strength (or weakness) indicated by a value of <30 kg, preferably <26 kg, for men or <20 kg, preferably <16 kg, for women in the handgrip strength test.
16. A myostatin or activin antagonist for use according to any one of the aspects 1 to 12 wherein sarcopenia is defined by at least one of the criteria of low physical performance as defined in aspect 13 and by at least one of the criteria of low muscle mass as defined in aspect 14.
17. A myostatin or activin antagonist for use according to any one of the aspects 1 to 12 wherein sarcopenia is defined by at least one of the criteria of low muscle mass as defined in aspect 14 and by the criteria of low muscle strength as defined in aspect 15.
18. A myostatin or activin antagonist for use according to any one of the aspects 1 to 12 wherein sarcopenia is defined by at least one of the criteria of low physical performance as defined in aspect 13 and by the criteria of low muscle strength as defined in aspect 15.
19. A myostatin or activin antagonist for use according to any one of the aspects 1 to 12 wherein sarcopenia is defined by at least one of the criteria of low physical performance as defined in aspect 13, and by at least one of the criteria of low muscle mass as defined in aspect 14, and by the criteria of low muscle strength as defined in aspect 15.

In a preferred embodiment the present invention provides the myostatin or activin antagonist bimagrumab for use according to any one of the aspects 1 to 12 wherein sarcopenia is defined by the criteria of low muscle mass as indicated by an AL(B)M adjusted for body mass index (BMI) of ≦0.789 kg for men or ≦0.512 kg for women, said AL(B)M being measured by dual energy X-ray absorptiometry (DXA) and by the criteria of low muscle strength as indicated by a value of <26 kg for men or <16 kg for women in the handgrip strength test.

In another preferred embodiment the present invention provides the myostatin or activin antagonist bimagrumab for use according to any one of the aspects 1 to 12 wherein sarcopenia is defined by the criteria of low muscle mass as indicated by an appendicular skeletal muscle index (ASMI) of ≦7.26 kg/m² for men or ≦5.5 kg/m² for women, said ASMI being defined as appendicular skeletal muscle mass divided by the square of height, said ASMI being measured by dual energy X-ray absorptiometry (DXA) and by the criteria of low muscle strength as indicated by a value of <30 kg for men or <20 kg for women in the handgrip strength test.

In another preferred embodiment the present invention provides the myostatin or activin antagonist bimagrumab for use according to any one of the aspects 1 to 12 wherein sarcopenia is defined by the criteria of low physical performance (or mobility limitations) indicated by a gait speed over a 4-m course of ≦1 m/s, preferably <0.8 m/s, and by the criteria of low muscle mass as indicated by an appendicular skeletal muscle index (ASMI) of ≦7.26 kg/m² for men or ≦5.5 kg/m² for women, said ASMI being defined as appendicular skeletal muscle mass divided by the square of height, said ASMI being measured by dual energy X-ray absorptiometry (DXA).

In another preferred embodiment the present invention provides the myostatin or activin antagonist bimagrumab for use according to any one of the aspects 1 to 12 wherein sarcopenia is defined by a gait speed over a 4-m course of >0.8 m/s, and by a value of <30 kg for men or <20 kg for women in the handgrip strength test, and an appendicular skeletal muscle index (ASMI) of 7.26 kg/m² for men or 5.5 kg/m² for women, said ASMI being defined as appendicular skeletal muscle mass divided by the square of height, said ASMI being measured by dual energy X-ray absorptiometry (DXA).

In another preferred embodiment the present invention provides the myostatin or activin antagonist bimagrumab for use according to any one of the aspects 1 to 12 wherein sarcopenia is defined by a gait speed over a 4-m course of <0.8 m/s, and an appendicular skeletal muscle index (ASMI) of 7.26 kg/m² for men or 5.5 kg/m² for women, said ASMI being defined as appendicular skeletal muscle mass divided by the square of height, said ASMI being measured by dual energy X-ray absorptiometry (DXA).
20. Pharmaceutical composition comprising bimagrumab for use according to any one of the aspects 4 to 19 wherein said composition is provided as a concentrated aqueous solution and wherein the concentration of bimagrumab is from 100 to 200 mg/mL, preferably 135 to 165 mg/mL, more preferably ca. 150 mg/mL.
21. Pharmaceutical composition according to aspect 20 wherein said concentrated aqueous solution is diluted for intraveneous administration with an isotonic aqueous solution, preferably 5% dextrose, and wherein the concentration of bimagrumab in the diluted solution is from 0.2 to 10 mg/mL.
22. Pharmaceutical composition according to aspect 21 wherein said diluted solution is intraveneously administered with an infusion flow rate of 1-10 mL/min, preferably 2-4 mL/min.
23. Bimagrumab for use in treating age related sarcopenia, wherein bimagrumab is administered intravenously at a dose regimen of about 70 mg once every 4 weeks.
24. Bimagrumab for use in treating age related sarcopenia, wherein bimagrumab is administered intravenously at a dose regimen of about 210 mg once every 4 weeks.
25. Bimagrumab for use in treating age related sarcopenia, wherein bimagrumab is administered intravenously at a dose regimen of about 700 mg once every 4 weeks.

The present disclosure also comprise the use of a myostatin or activin antagonists according to any preceding aspect (including dosing, dosing regimen, intervals of administration and specific patients and end points) for the manufacture of a medicament for the treatment of sarcopenia, physical frailty, frailty, or physical frailty & sarcopenia.

The present disclosure also comprise the use of a myostatin or activin antagonists according to any preceding aspect (including dosing, dosing regimen, intervals of administration and specific patients and end points) for the manufacture of a medicament for the treatment of sarcopenia, physical frailty, frailty, or physical frailty & sarcopenia.

The present disclosure also comprise methods of treating sarcopenia, physical frailty, frailty or physical frailty & sarcopenia comprising administering a myostatin or activin antagonists according to any preceding aspect (including dosing, dosing regimen, intervals of administration and specific patients and end points).

Every aspect, method or use can be combined with each other within the scope of the present disclosure.

The manufacture of bimagrumab has been described in WO2010/125003.

Bimagrumab comprises an antigen binding site comprising at least one immunoglobulin heavy chain variable domain (V_{H}) which comprises in sequence hypervariable regions CDR1 of SEQ ID No 1, CDR2 of SEQ ID No 2 and CDR3 of SEQ ID No 3.

The use of antibodies having 1, 2 or 3 residues changed from any of the sequences of CDR1, CDR2 and/or CDR3 of the heavy chain is also comprised within the scope of the invention.

Bimagrumab also comprises antigen binding site comprising at least one immunoglobulin light chain variable domain (V_{L}) which comprises in sequence hypervariable regions CDR1 of SEQ ID No 4, CDR2 of SEQ ID No 5 and CDR3 of SEQ ID No 6 or CDR equivalents thereof. The use of antibodies having 1, 2 or 3 residues changed from any of the sequences of CDR1, CDR2 and/or CDR3 of the light chain is also comprised within the scope of the invention.

Bimagrumab also comprises a light chain of SEQ ID No 7 or SEQ ID No 8 and a heavy chain of SEQ ID No 9.

According to the invention the use of antibodies having 95% identity with the light chain and/or the heavy chain are also comprised.

The terms “sarcopenia”, “frailty”, “physical frailty”, “physical frailty & sarcopenia” according to the present invention are all generally defined as low muscle mass and impaired mobility. The term “treatment of sarcopenia” or treatment of frailty”, physical frailty, physical frailty & sarcopenia therefore comprise the improvement of mobility and the reduction of the risk of falls. In particular the treatment of sarcopenia comprises the risk of injurious falls or falls leading to hospitalization and is indicated to preserve independence.

The term “sarcopenia” and other terms such as “frailty”, “physical frailty”, “physical frailty & sarcopenia” according to the present invention are also defined by the following alternative definitions:
1. Sarcopenia is defined by the criterion of low physical performance (or mobility limitations) indicated by at least one of the following:
   (a) a walking distance of <400 m in the 6 minute walk test (6MWT);
   (b) a time of >15 min in the 400 m walk test;
   (c) a short physical performance battery (SPPB) score of ≦8;
   (d) a gait speed over a 4-m course of ≦1 m/s, preferably <0.8 m/s, more preferably <0.8 m/s or ≦0.8 ms but ≧0.3 m/s.
2. Sarcopenia is defined by the criterion of low muscle mass (or low skeletal muscle mass) indicated by at least one of the following:
   (a) a appendicular skeletal muscle index (ASMI) of ≦7.26 kg/m² for men or ≦5.5 kg/m² for women, said ASMI being defined as appendicular skeletal muscle mass divided by the square of height;
   (b) an appendicular lean (body) mass (AL(B)M) of ≦19.75 kg for men or ≦15.02 kg for women;
   (c) an AL(B)M adjusted for body mass index (BMI) of 0.789 kg for men or ≦0.512 kg for women;
   said ASMI and AL(B)M being measured by dual energy X-ray absorptiometry (DXA) and said TMV being measured by magnetic resonance imaging (MRI).
3. Sarcopenia is defined by the criterion of low muscle strength (or weakness) indicated by a value of <30 kg, preferably <26 kg, for men or <20 kg, preferably <16 kg, for women in the handgrip strength test.

The term “sarcopenia” according to the present invention is preferably defined by the following alternative definitions:
4. Sarcopenia is defined by at least one of the criteria of low physical performance as defined in definition 1 and by at least one of the criteria of low muscle mass as defined in definition 2.
5. Sarcopenia is defined by at least one of the criteria of low muscle mass as defined in definition 2 and by the criteria of low muscle strength as defined in definition 3.
6. Sarcopenia is defined by at least one of the criteria of low physical performance as defined in definition 1 and by the criteria of low muscle strength as defined in definition 3.
7. Sarcopenia is defined by at least one of the criteria of low physical performance as defined in definition 1, and by at least one of the criteria of low muscle mass as defined in definition 2, and by the criteria of low muscle strength as defined in definition 3.

In a particularly preferred definition of sarcopenia according to the present invention sarcopenia is defined by the criteria of low muscle mass as indicated by an AL(B)M adjusted for body mass index (BMI) of ≦0.789 kg for men or ≦0.512 kg for women, said AL(B)M being measured by dual energy X-ray absorptiometry (DXA) and by the criteria of low muscle strength as indicated by a value of <26 kg for men or <16 kg for women in the handgrip strength test.

In another particularly preferred definition of sarcopenia according to the present invention sarcopenia is defined by the criteria of low muscle mass as indicated by an appendicular skeletal muscle index (ASMI) of 7.26 kg/m² for men or 5.5 kg/m² for women, said ASMI being defined as appendicular skeletal muscle mass divided by the square of height, said ASMI being measured by dual energy X-ray absorptiometry (DXA) and by the criteria of low muscle strength as indicated by a value of <30 kg for men or <20 kg for women in the handgrip strength test.

In another particularly preferred definition of sarcopenia according to the present invention sarcopenia is defined by the criteria of low physical performance (or mobility limitations) indicated by a gait speed over a 4-m course of 1 m/s, preferably <0.8 m/s, and by the criteria of low muscle mass as indicated by an appendicular skeletal muscle index (ASMI) of ≦7.26 kg/m² for men or 5.5 kg/m² for women, said ASMI being defined as appendicular skeletal muscle mass divided by the square of height, said ASMI being measured by dual energy X-ray absorptiometry (DXA).

In another particularly preferred definition of sarcopenia according to the present invention sarcopenia is defined by a gait speed over a 4-m course of >0.8 m/s, and by a value of <30 kg for men or <20 kg for women in the handgrip strength test, and an appendicular skeletal muscle index (ASMI) of ≦7.26 kg/m² for men or ≦5.5 kg/m² for women, said ASMI being defined as appendicular skeletal muscle mass divided by the square of height, said ASMI being measured by dual energy X-ray absorptiometry (DXA).

In another particularly preferred definition of sarcopenia according to the present invention sarcopenia is defined by a gait speed over a 4-m course of 0.8 m/s, and an appendicular skeletal muscle index (ASMI) of ≦7.26 kg/m² for men or ≦5.5 kg/m² for women, said ASMI being defined as appendicular skeletal muscle mass divided by the square of height, said ASMI being measured by dual energy X-ray absorptiometry (DXA).

### EXAMPLES

Hereinafter, the present invention is described in more details and specifically with reference to the examples, which however are not intended to limit the present invention.

### Example 1

### Proof of Concept Study in Sarcopenic Adults With Mobility Limitations Treated With BYM338 (Bimagrumab)

A randomized, double-blind, placebo-controlled multi-center (USA, five centers) study was performed to assess the effects of BYM338 on skeletal muscle in sarcopenic adults with mobility limitations.

Primary Objectives:
Characterize the pharmacodynamic (PD) effect of BYM338 administered as i.v. infusions on muscle volume of the thigh (assessed by MRI) in sarcopenic adults with mobility limitations as compared to placebo.
Evaluate the effect of BYM338 on gait speed in these older patients.

Secondary Objectives:
Assess the safety and tolerability of BYM338 administered as i.v. infusions to older adults with sarcopenia.
Determine the pharmacokinetic (PK) profile of infusions of BYM338 in the older population with low muscle mass.

Test Product(s), Dose(s), and Mode(s) of Administration:

30 mg/kg, administered as i.v. infusion, delivered as liquid in vial with 150 mg BYM338 dose per vial.

Statistical Methods:

The primary objective was to assess the preliminary efficacy of one or two i.v. doses of BYM338 to increase mid-thigh muscle volume and gait speed compared to placebo.

The primary endpoints were change in TMV by MRI from baseline in patients receiving BYM338 compared to placebo at 8 weeks (for the interim analysis) and gait speed at 16 weeks post-first dose in terms of ratio post-baseline to baseline.

Primary endpoints were also assessed at 2 (only for muscle mass increase), 4 and 20 (only for gait speed) and 24 weeks, to document any decline in both outcomes beyond week 16. The choice of the 8 week time-point was driven by the assumption that a measurable post-dose effect of BYM338 on TMV was likely to be observed at that time, while 16 weeks were necessary to achieve a clinically significant effect on gait speed.

In order to compare the BYM338 group versus the Placebo group, an analysis of covariance model was performed on the log scale for the muscle mass assessed by MRI. Values were back transformed with exponential transformation to estimate the LS means of ratio to baseline. The analysis of covariance models included the treatment and the baseline (log transformed). P values were provided for the ratio to baseline at each time point and for the comparison between the BYM338 group and the placebo group. For each time point, values were considered independently. Least square means for each treatment group was calculated with the corresponding 90% confidence intervals, as well as differences vs. placebo.

For the gait speed, the same model was applied on the absolute change from baseline, with the treatment and the baseline as covariates. No back transformation was needed. The same results as with the model on the log scale for muscle mass assessed by MRI were provided. For gait speed, the same model was also applied on the values stratified by score at baseline: high value at baseline (>=0.8 m/s) and low value at baseline (<0.8 m/s).

The same analysis of covariance used to describe the muscle volume by MRI was also performed on parameters assessed by DXA (lean body mass), grip strength, stair climbing, 1-RM leg press and physical activity monitoring (ActivPAL).

For the 6 minute walk test, the same model used for gait speed (on the absolute change from baseline) was performed. A stratified analysis was also performed according to the baseline value: High value (>=300 m) and Low value (<300 m).

Descriptive statistics of PK parameters included mean, SD, and CV, min and max. When a geometric mean was presented it was stated as such. Since Tmax is generally evaluated by a nonparametric method, median values and ranges were given for this parameter.

No exploratory analyses to investigate the relationship between exposure and primary PD endpoints were carried out.

### Study Population: Key Inclusion/Exclusion Criteria

Diagnosis and Main Criteria for Inclusion:

Key criteria to qualify for this study include:
1. Men and women aged 65 or older with difficulty standing up from a chair or walking for longer than 10 minutes on a flat surface or climbing a flight of stairs.
2. A gait speed over 4 meters of <1.0 m/s but ≧0.4 m/s.
3. Appendicular skeletal muscle index (skeletal muscle in kg/height in m2) by DXA of ≦7.25 kg/m2 for men and ≦5.67 kg/m2 for women.
4. Patients had to weigh between 40 and 120 kg and have a body mass index (BMI) within the range of 18-32 kg/m2.

Exclusion Criteria:
1. Use of other investigational drugs at the time of enrollment, or within 30 days or 5 half-lives of enrollment, whichever was longer; or longer if required by local regulations, and for any other limitation of participation in an investigational trial based on local regulations.
2. History of hypersensitivity to antibody therapy.
3. A history of clinically significant ECG abnormalities, which, in the opinion of the investigator, might indicate active cardiac disease.
4. History of malignancy of any organ system (other than localized basal cell carcinoma of the skin), treated or untreated, within the past 5 years, regardless of whether there was evidence of local recurrence or metastases.
5. Diseases other than cancer known to cause cachexia or muscle atrophy, including but was not limited to congestive heart failure, COPD, chronic kidney disease (estimated GFR<30 mL/min using the MDRD equation), rheumatoid arthritis, primary myopathy, stroke, HIV infection, tuberculosis or other chronic infection, uncontrolled diabetes mellitus, etc.
6. Diseases known to cause malabsorption of protein or energy, including inflammatory bowel disease, celiac disease, short bowel syndrome, pancreatic insufficiency, etc.
7. Liver disease or liver injury as indicated by abnormal liver function tests such as SGOT (AST), SGPT (ALT), γ-GT, alkaline phosphatase, or serum bilirubin (except Gilbert's Disease). The Investigator was guided by the following criteria:
   Any single transaminase listed above was not to exceed 3×upper limit of normal (ULN).
   If the total bilirubin concentration was increased above 1.5×ULN, total bilirubin was required to be differentiated into the direct and indirect reacting bilirubin. In any case, serum bilirubin was not to exceed the value of 1.6 mg/dL (27 μmol/L).
8. Use of any prescription drugs known to affect muscle mass, including androgen supplements, anti-androgens (such as LHRH agonists), recombinant human growth hormone (rhGH), insulin, oral beta agonists, megestrol acetate, dronabinol, etc.
9. Donation or loss of 400 ml or more of blood within eight weeks prior to initial dosing, or longer if required by local regulation.
10. Plasma donation (>250 ml) within 14 days prior to first dosing.
11. Hemoglobin levels below 11.0 g/dL at screening.
12. Significant illness within two weeks prior to dosing.
13. Recent (within the last three years) and/or recurrent history of autonomic dysfunction (e.g., recurrent episodes of fainting, palpitations, etc.).
14. Patients with known claustrophobia, presence of pacemaker and/or ferromagnetic material in their body that would prohibit administration of MRI assessments
15. Patient smokes more than one cigarette, pipe or cigar a month

### Participant Flow Table

Subject Disposition−N (Percent) of Patients (All Patients)

| | 30 mg/kg | | |
|---|---|---|---|
| | BYM338 | Placebo | Total |
| | N = 19 | N = 21 | N = 40 |
| Patients | | | |
| Completed | 15 (78.9%) | 17 (81.0%) | 32 (80.0%) |
| Discontinued | 3 (15.8%) | 2 (9.5%) | 5 (12.5%) |
| Withdrew due to Adverse Event(s) | 1 (5.3%) | | 1 (2.5%) |
| Lost to follow-up | 1 (5.3%) | | 1 (2.5%) |
| Subject withdrew consent | 1 (5.3%) | 2 (9.5%) | 3 (7.5%) |
| Missing *EOS visit data | 1 (5.3%) | 2 (9.5%) | 3 (7.5%) |

*Three patients (BYM338: 1003/5104; Placebo: 1002/5141 and 1003/5109) are missing study completion data

### Baseline Characteristics

Demographic Summary (Safety Analysis Set)

| | | 30 mg/kg | | |
|---|---|---|---|---|
| | | BYM338 | Placebo | Total |
| | | N = 19 | N = 21 | N = 40 |
| Age (years) | Mean (SD) | 71.6 (6.34) | 72.4 (4.62) | 72.0 (5.45) |
| | Median | 69.0 | 73.0 | 73.0 |
| | Range | 65-86 | 65-83 | 65-86 |
| Gender- | Male | 13 (68%) | 8 (38%) | 21 (53%) |
| n(%) | Female | 6 (32%) | 13 (62%) | 19 (48%) |
| Predominant | Caucasian | 18 (95%) | 21 (100%) | 39 (98%) |
| race-n(%) | Black | 1 (5%) | | 1 (3%) |
| Ethnicity- | Hispanic/ | 15 (79%) | 13 (62%) | 28 (70%) |
| n(%) | Latino | | | |
| | Other | 4 (21%) | 8 (38%) | 12 (30%) |
| Height | Mean (SD) | 166.5 (9.3) | 165.2 (8.7) | 165.8 (8.9) |
| (cm) | Median | 167.6 | 163.0 | 164.5 |
| | Range | 144.0-182.0 | 152.0-185.0 | 144.0-185.0 |
| Weight | Mean (SD) | 69.0 (10.8) | 71.4 (10.3) | 70.3 (10.5) |
| (kg) | Median | 70.9 | 68.2 | 69.6 |
| | Range | 47.7-91.4 | 55.1-100.0 | 47.7-100.0 |
| BMI | Mean (SD) | 24.9 (3.7) | 26.2 (3.5) | 25.6 (3.6) |
| (kg/m²) | Median | 25.2 | 25.8 | 25.7 |
| | Range | 18.0-30.9 | 19.1-32.0 | 18.0-32.0 |

### Summary of Efficacy

### Primary Outcome Result(s)

Total Thigh Muscle Volume—Percentage Change From Baseline (PD Analysis Set)

| | | % change | % change | % change | % change |
|---|---|---|---|---|---|
| | | from | from | from | from |
| | | Baseline (%) | Baseline (%) | Baseline (%) | Baseline (%) |
| Treatment | Statistic | W4D29 | W8D57 | W16D113 | EOS |
| 30 mg/kg | n | 17 | 17 | 16 | 14 |
| BYM338 | Mean (SD) | 6.1 (2.6) | 8.0 (3.7) | 7.7 (5.3) | 4.8 (5.8) |
| | CV % mean | 41.9 | 46.2 | 68.8 | 121.1 |
| | Median | 5.9 | 8.3 | 7.7 | 4.5 |
| | Min-max | 2.1-10.8 | 0.73-15.7 | 0.015-17.6 | −3.9-15.0 |
| Placebo | n | 19 | 18 | 17 | 16 |
| | Mean (SD) | 0.16 (3.4) | 0.35 (3.3) | 0.42 (5.1) | −1.01 (4.4) |
| | CV % mean | 2151.1 | 955.7 | 1224.4 | −437.3 |
| | Median | 0.54 | 1.11 | 1.21 | −0.05 |
| | Min-max | −9.39-5.93 | −10.2-5.9 | −16.3-6.7 | −15.9-2.8 |

CV % = coefficient of variation (%) = sd/mean*100;
Baseline is Day −1 value

ANCOVA Results on Gait Speed Abs Change (M/S) From Baseline—Stratified by Baseline Score—Pharmacodynamic Analysis Set

| Status at | | Treatment/ | Number of | P | | 90% CI | |
|---|---|---|---|---|---|---|---|
| Baseline | Visit | contrast | patients | value | Estimate | Lower | Upper |
| High Value | W2D15 | 30 mg/kg BYM338 | 10 | | 0.05 | −0.04 | 0.13 |
| at Baseline | | Placebo | 12 | | −0.00 | −0.07 | 0.07 |
| (>= 0.8 m/s) | | Difference 30 mg/kg | | 0.501 | 0.05 | −0.07 | 0.16 |
| | | BYM338 vs Placebo | | | | | |
| | W4D29 | 30 mg/kg BYM338 | 9 | | 0.06 | 0.01 | 0.12 |
| | | Placebo | 12 | | 0.03 | −0.02 | 0.07 |
| | | Difference 30 mg/kg | | 0.442 | 0.03 | −0.04 | 0.11 |
| | | BYM338 vs Placebo | | | | | |
| | W6D43 | 30 mg/kg BYM338 | 9 | | 0.10 | 0.02 | 0.19 |
| | | Placebo | 12 | | 0.03 | −0.04 | 0.10 |
| | | Difference 30 mg/kg | | 0.304 | 0.07 | −0.05 | 0.19 |
| | | BYM338 vs Placebo | | | | | |
| | W8D57 | 30 mg/kg BYM338 | 9 | | 0.13 | 0.05 | 0.20 |
| | | Placebo | 12 | | 0.06 | −0.01 | 0.12 |
| | | Difference 30mg/kg | | 0.232 | 0.07 | −0.03 | 0.17 |
| | | BYM338 vs Placebo | | | | | |
| | W10D71 | 30 mg/kg BYM338 | 9 | | 0.15 | 0.08 | 0.22 |
| | | Placebo | 12 | | 0.07 | 0.01 | 0.13 |
| | | Difference 30 mg/kg | | 0.194 | 0.08 | −0.02 | 0.18 |
| | | BYM338 vs Placebo | | | | | |
| | W12D85 | 30 mg/kg BYM338 | 9 | | 0.12 | 0.02 | 0.22 |
| | | Placebo | 12 | | 0.10 | 0.02 | 0.22 |
| | | Difference 30 mg/kg | | 0.851 | 0.02 | −0.12 | 0.15 |
| | | BYM338 vs Placebo | | | | | |
| | W16D113 | 30 mg/kg BYM338 | 8 | | 0.11 | 0.01 | 0.22 |
| | | Placebo | 11 | | 0.09 | 0.00 | 0.18 |
| | | Difference 30 mg/kg | | 0.786 | 0.02 | −0.12 | 0.16 |
| | | BYM338 vs Placebo | | | | | |
| | W20D141 | 30 mg/kg BYM338 | 9 | | 0.19 | 0.11 | 0.27 |
| | | Placebo | 11 | | 0.14 | 0.07 | 0.22 |
| | | Difference 30 mg/kg | | 0.449 | 0.05 | −0.06 | 0.17 |
| | | BYM338 vs Placebo | | | | | |
| | EOS | 30 mg/kg BYM338 | 9 | | 0.17 | 0.08 | 0.26 |
| | | Placebo | 11 | | 0.11 | 0.03 | 0.19 |
| | | Difference 30 mg/kg | | 0.401 | 0.06 | −0.06 | 0.19 |
| | | BYM338 vs Placebo | | | | | |
| Low Value | W2D15 | 30 mg/kg BYM338 | 8 | | 0.23 | 0.10 | 0.35 |
| at Baseline | | Placebo | 8 | | 0.28 | 0.15 | 0.41 |
| (<0.8 m/s) | | Difference 30 mg/kg | | 0.599 | −0.05 | −0.23 | 0.12 |
| | | BYM338 vs Placebo | | | | | |
| | W4D29 | 30 mg/kg BYM338 | 9 | | 0.29 | 0.18 | 0.40 |
| | | Placebo | 8 | | 0.21 | 0.09 | 0.33 |
| | | Difference 30 mg/kg | | 0.389 | 0.08 | −0.08 | 0.25 |
| | | BYM338 vs Placebo | | | | | |
| | W6D43 | 30 mg/kg BYM338 | 9 | | 0.29 | 0.15 | 0.43 |
| | | Placebo | 8 | | 0.27 | 0.12 | 0.41 |
| | | Difference 30 mg/kg | | 0.853 | 0.02 | −0.18 | 0.22 |
| | | BYM338 vs Placebo | | | | | |
| | W8D57 | 30 mg/kg BYM338 | 8 | | 0.36 | 0.27 | 0.46 |
| | | Placebo | 8 | | 0.38 | 0.28 | 0.47 |
| | | Difference 30mg/kg | | 0.824 | −0.02 | −0.15 | 0.12 |
| | | BYM338 vs Placebo | | | | | |
| | W10D71 | 30 mg/kg BYM338 | 8 | | 0.43 | 0.35 | 0.51 |
| | | Placebo | 7 | | 0.36 | 0.28 | 0.45 |
| | | Difference 30 mg/kg | | 0.328 | 0.07 | −0.05 | 0.19 |
| | | BYM338 vs Placebo | | | | | |
| | W12D85 | 30 mg/kg BYM338 | 8 | | 0.34 | 0.24 | 0.43 |
| | | Placebo | 7 | | 0.30 | 0.20 | 0.41 |
| | | Difference 30 mg/kg | | 0.686 | 0.03 | −0.11 | 0.18 |
| | | BYM338 vs Placebo | | | | | |
| | W16D113 | 30 mg/kg BYM338 | 8 | | 0.50 | 0.44 | 0.56 |
| | | Placebo | 7 | | 0.35 | 0.28 | 0.41 |
| | | Difference 30 mg/kg | | 0.009 | 0.15 | 0.06 | 0.24 |
| | | BYM338 vs Placebo | | | | | |
| | W20D141 | 30 mg/kg BYM338 | 8 | | 0.51 | 0.39 | 0.64 |
| | | Placebo | 7 | | 0.40 | 0.27 | 0.54 |
| | | Difference 30 mg/kg | | 0.310 | 0.11 | −0.08 | 0.30 |
| | | BYM338 vs Placebo | | | | | |
| | EOS | 30 mg/kg BYM338 | 7 | | 0.42 | 0.30 | 0.55 |
| | | Placebo | 7 | | 0.37 | 0.25 | 0.49 |
| | | Difference 30 mg/kg | | 0.604 | 0.05 | −0.12 | 0.23 |
| | | BYM338 vs Placebo | | | | | |

CI: Confidence Interval
Model: Change from baseline = Treatment + Baseline
Baseline: Last value before first treatment dose (Screening or Day −1).

### Secondary Outcome Result(s)

Summary Statistics for PK Parameters (Pharmacokinetic Analysis Set)

| | | Cmax | Tmax | AUC0-56 | AUClast |
|---|---|---|---|---|---|
| Dose | Statistic | (μg/mL) | (hr) | (day*μg/mL) | (day*μg/mL) |
| 1 | n | 19 | 19 | 16 | 7 |
| | Mean (SD) | 707 (118) | | 6060 (1100) | 6550 (870) |
| | CV % mean | 16.8 | | 18.2 | 13.3 |
| | Geo-mean | 697 | | 5970 | 6500 |
| | CV % geo-mean | 17.1 | | 18.3 | 13.2 |
| | Median | 702 | 2.57 | 5970 | 6610 |
| | (min/max) | 530-903 | 1.83-6.28 | 3840-8940 | 5500-7820 |
| 2 | n | 9 | 9 | 9 | 9 |
| | Mean (SD) | 808 (162) | | 9130 (1770) | 9690 (2110) |
| | CV % mean | 20.0 | | 19.4 | 21.8 |
| | Geo-mean | 794 | | 8990 | 9500 |
| | CV % geo-mean | 20.3 | | 19.0 | 20.8 |
| | Median | 812 | 2.15 | 9150 | 9590 |
| | (min/max) | 595-1060 | 2.08-2.32 | 6550-12900 | 6870-14400 |

CV % = coefficient of variation (%) = sd/mean*100;
CV % geo-mean = (sqrt (exp. (variance for log transformed data)-1))*100

### Summary of Safety

### Safety Results

Adverse Events Overall and Frequently Affected System Organ Classes−N (Percent) of Patients (Safety Analysis Set)

| | 30 mg/kg | | |
|---|---|---|---|
| | BYM338 | Placebo | Total |
| | N = 19 | N = 21 | N = 40 |
| | n (%) | n (%) | n (%) |
| Patients with AE(s) | 16 (84.2%) | 12 (57.1%) | 28 (70.0%) |
| Musculoskeletal and connective | 13 (68.4%) | 8 (38.1%) | 21 (52.5%) |
| tissue disorders | | | |
| Nervous system disorders | 4 (21.1%) | 4 (19.0%) | 8 (20.0%) |
| Gastrointestinal disorders | 6 (31.6%) | 2 (9.5%) | 8 (20.0%) |
| Infections and infestations | 5 (26.3%) | 2 (9.5%) | 7 (17.5%) |
| Skin and subcutaneous | 3 (15.8%) | 2 (9.5%) | 5 (12.5%) |
| tissue disorders | | | |
| Investigations | 2 (10.5%) | 2 (9.5%) | 4 (10.0%) |
| Respiratory, thoracic and | 1 (5.3%) | 1 (4.8%) | 2 (5.0%) |
| mediastinal disorders | | | |
| General disorders and | 1 (5.3%) | 1 (4.8%) | 2 (5.0%) |
| administration site | | | |
| conditions | | | |
| Vascular disorders | 1 (5.3%) | 0 | 1 (2.5%) |
| Reproductive system and breast | 0 | 1 (4.8%) | 1 (2.5%) |
| disorders | | | |
| Injury, poisoning and procedural | 0 | 1 (4.8%) | 1 (2.5%) |
| complications | | | |
| Immune system disorders | 1 (5.3%) | 0 | 1 (2.5%) |
| Ear and labyrinth disorders | 0 | 1 (4.8%) | 1 (2.5%) |
| Cardiac disorders | 1 (5.3%) | 0 | 1 (2.5%) |
| Blood and lymphatic system | 1 (5.3%) | 0 | 1 (2.5%) |
| disorders | | | |

Adverse Events−N (Percent) of Patients (All Patients)—Safety Analysis Set

| | 30 mg/kg | | |
|---|---|---|---|
| | BYM338 | Placebo | Total |
| | N = 19 | N = 21 | N = 40 |
| | n (%) | n (%) | n (%) |
| Patients with AE(s) | 16 (84.2%) | 12 (57.1%) | 28 (70.0%) |
| Muscle spasms | 9 (47.4%) | 4 (19.0%) | 13 (32.5%) |
| Muscle twitching | 3 (15.8%) | 1 (4.8%) | 4 (10.0%) |
| Limb discomfort | 2 (10.5%) | 2 (9.5%) | 4 (10.0%) |
| Diarrhoea | 4 (21.1%) | 0 | 4 (10.0%) |
| Pain in extremity | 1 (5.3%) | 2 (9.5%) | 3 (7.5%) |
| Vomiting | 2 (10.5%) | 0 | 2 (5.0%) |
| Skin exfoliation | 0 | 2 (9.5%) | 2 (5.0%) |
| Paraesthesia | 1 (5.3%) | 1 (4.8%) | 2 (5.0%) |
| Myalgia | 0 | 2 (9.5%) | 2 (5.0%) |
| Muscle tightness | 1 (5.3%) | 1 (4.8%) | 2 (5.0%) |
| Hypoaesthesia | 1 (5.3%) | 1 (4.8%) | 2 (5.0%) |
| Headache | 1 (5.3%) | 1 (4.8%) | 2 (5.0%) |
| Blood pressure increased | 1 (5.3%) | 1 (4.8%) | 2 (5.0%) |
| Blood creatine phosphokinase increased | 1 (5.3%) | 1 (4.8%) | 2 (5.0%) |
| Back pain | 1 (5.3%) | 1 (4.8%) | 2 (5.0%) |
| Acne | 2 (10.5%) | 0 | 2 (5.0%) |
| Vertigo | 0 | 1 (4.8%) | 1 (2.5%) |
| Urinary tract infection | 0 | 1 (4.8%) | 1 (2.5%) |
| Upper respiratory tract infection | 0 | 1 (4.8%) | 1 (2.5%) |
| Tooth loss | 1 (5.3%) | 0 | 1 (2.5%) |
| Soft tissue injury | 0 | 1 (4.8%) | 1 (2.5%) |
| Soft tissue disorder | 0 | 1 (4.8%) | 1 (2.5%) |
| Skin fissures | 0 | 1 (4.8%) | 1 (2.5%) |
| Sinus arrhythmia | 1 (5.3%) | 0 | 1 (2.5%) |
| Rhinitis allergic | 1 (5.3%) | 0 | 1 (2.5%) |
| Respiratory tract infection | 1 (5.3%) | 0 | 1 (2.5%) |
| Rash pustular | 1 (5.3%) | 0 | 1 (2.5%) |
| Pruritus | 0 | 1 (4.8%) | 1 (2.5%) |
| Perineal abscess | 1 (5.3%) | 0 | 1 (2.5%) |
| Papule | 1 (5.3%) | 0 | 1 (2.5%) |
| Neck pain | 1 (5.3%) | 0 | 1 (2.5%) |
| Nausea | 1 (5.3%) | 0 | 1 (2.5%) |
| Nasal congestion | 0 | 1 (4.8%) | 1 (2.5%) |
| Musculoskeletal discomfort | 0 | 1 (4.8%) | 1 (2.5%) |
| Musculoskeletal chest pain | 1 (5.3%) | 0 | 1 (2.5%) |
| Muscular weakness | 1 (5.3%) | 0 | 1 (2.5%) |
| Muscle fatigue | 0 | 1 (4.8%) | 1 (2.5%) |
| Malaise | 1 (5.3%) | 0 | 1 (2.5%) |
| Intracranial venous sinus thrombosis | 1 (5.3%) | 0 | 1 (2.5%) |
| Infusion site reaction | 0 | 1 (4.8%) | 1 (2.5%) |
| Hypertension | 1 (5.3%) | 0 | 1 (2.5%) |
| Herpes zoster | 1 (5.3%) | 0 | 1 (2.5%) |
| Hair growth abnormal | 1 (5.3%) | 0 | 1 (2.5%) |
| Gingival inflammation | 0 | 1 (4.8%) | 1 (2.5%) |
| Gastro sophageal reflux disease | 1 (5.3%) | 0 | 1 (2.5%) |
| Fibrocystic breast disease | 0 | 1 (4.8%) | 1 (2.5%) |
| Fall | 0 | 1 (4.8%) | 1 (2.5%) |
| Erythema | 0 | 1 (4.8%) | 1 (2.5%) |
| Drug hypersensitivity | 1 (5.3%) | 0 | 1 (2.5%) |
| Dizziness | 0 | 1 (4.8%) | 1 (2.5%) |
| Bronchitis | 1 (5.3%) | 0 | 1 (2.5%) |
| Arthralgia | 1 (5.3%) | 0 | 1 (2.5%) |
| Anaemia | 1 (5.3%) | 0 | 1 (2.5%) |
| Abdominal pain upper | 0 | 1 (4.8%) | 1 (2.5%) |
| Abdominal pain | 1 (5.3%) | 0 | 1 (2.5%) |

Arranged by frequency in the total column

Adverse Events−N (Percent) of Patients (All Patients)—Suspected Treatment Related (Safety Analysis Set)

| | 30 mg/kg | | |
|---|---|---|---|
| | BYM338 | Placebo | Total |
| | N = 19 | N = 21 | N = 40 |
| | n (%) | n (%) | n (%) |
| Patients with AE(s) | 13 (68.4%) | 6 (28.6%) | 19 (47.5%) |
| Muscle spasms | 9 (47.4%) | 4 (19.0%) | 13 (32.5%) |
| Muscle twitching | 2 (10.5%) | 1 (4.8%) | 3 (7.5%) |
| Myalgia | 0 | 2 (9.5%) | 2 (5.0%) |
| Muscle tightness | 1 (5.3%) | 1 (4.8%) | 2 (5.0%) |
| Hypoaesthesia | 1 (5.3%) | 1 (4.8%) | 2 (5.0%) |
| Skin exfoliation | 0 | 1 (4.8%) | 1 (2.5%) |
| Rash pustular | 1 (5.3%) | 0 | 1 (2.5%) |
| Paraesthesia | 1 (5.3%) | 0 | 1 (2.5%) |
| Papule | 1 (5.3%) | 0 | 1 (2.5%) |
| Pain in extremity | 0 | 1 (4.8%) | 1 (2.5%) |
| Muscle fatigue | 0 | 1 (4.8%) | 1 (2.5%) |
| Malaise | 1 (5.3%) | 0 | 1 (2.5%) |
| Limb discomfort | 0 | 1 (4.8%) | 1 (2.5%) |
| Infusion site reaction | 0 | 1 (4.8%) | 1 (2.5%) |
| Hair growth abnormal | 1 (5.3%) | 0 | 1 (2.5%) |
| Erythema | 0 | 1 (4.8%) | 1 (2.5%) |
| Diarrhea | 1 (5.3%) | 0 | 1 (2.5%) |
| Acne | 1 (5.3%) | 0 | 1 (2.5%) |
| Abdominal pain | 1 (5.3%) | 0 | 1 (2.5%) |

Arranged by frequency in the total column

### List of AEs—Skin and Subcutaneous

| | | | | | | Continued |
|---|---|---|---|---|---|---|
| | Center/ | Study | | | Relationship | beyond |
| Treatment | patient | Day | AE (preferred term) | Severity | to study drug | EOS visit |
| 30 mg/kg | 1001/5112 | 23 | Papule | Mild | Suspected | No |
| BYM338 | | 33 | Papule | Mild | Suspected | No |
| | 1002/5114 | 41 | Acne | Mild | Not suspected | No |
| | 1002/5114 | 150 | Hair growth abnormal | Mild | Suspected | No |
| | 1005/5134 | 43 | Acne | Moderate | Suspected | No |
| Placebo | 1004/5135 | 72 | Pruritus | Mild | Not suspected | No |
| | | 85 | Skin exfoliation | Mild | Suspected | No |
| | | | Erythema | Mild | Suspected | No |
| | 1005/5140 | 112 | Skin exfoliation | Mild | Not suspected | No |
| | | | Skin fissures | Mild | Not suspected | No |

### Conclusion:

One or two doses of BYM338 over 16 weeks was efficacious at increasing muscle mass in older adults with sarcopenia and promoting clinically meaningful improvements in physical function in patients with greater mobility disability. In addition, treatment with BYM338 was safe and well tolerated and resulted in a pharmacokinetic profile suggesting target mediated drug disposition with no treatment related immunogenicity signal, both consistent with prior studies with BYM338. Data from this study support the further evaluation of BYM338 in the older adult population with lower skeletal muscle mass and impaired physical function to bring about clinically meaningful improvement in functional capacity and a reduction in health risk and cost.

### Example 2

### Pharmacology, Toxicology, Pharmacokinetics and Pharmacodynamics

All information currently available on pharmacology, toxicology, pharmacokinetics, and pharmacodynamics has been obtained from in vitro experiments, animal trials, toxicology studies, and early human studies. To date, bimagrumab has been generally safe and well tolerated and efficacious at increasing muscle mass in adults 19-86 years of age. Findings and relevant data from prior studies are briefly described below.

### Human Safety and Tolerability Data

Approximately 450 adults have enrolled in early development clinical trials with bimagrumab with 155 adult men and women in six studies where data are available, having received single (n=134) or multiple (n=21) doses of active drug. Dose levels have ranged from 0.01 mg/kg to 30 mg/kg as i.v. infusions.

Study results to date indicate that bimagrumab is safe and well tolerated. The current safety profile is favorable, with adverse events to date limited to several minor clinical symptoms, two of which are being followed closely (acne and muscle spasms).

Transient cases of acne and periodic, involuntary muscle contractions of mostly mild intensity (referred to as “cramps or spasms”) have been observed in study participants to date with symptoms occurring more frequently in those subjects receiving the highest doses of drug (30 mg/kg). Several subjects have dropped out of earlier studies because of an AE (exacerbation of acne or muscle cramps). However, no one in the Proof of Concept study (CBYM338X2201) in subjects with sarcopenia and mobility limitations dropped out due to an AE.

Based on preclinical, toxicology and clinical findings to date the benefit/risk profile is positive and supports continued development in patients with skeletal muscle loss who would benefit from increased lean tissue.

### Human Pharmacokinetic Data

The PK of bimagrumab following single and repeat i.v. administrations show evidence of a similar nonlinear kinetics caused by target mediated drug disposition (TMDD) as described in the rat and cynomolgus monkey toxicology studies. Based on preliminary PK compartmental modeling, the loss of clearance saturation seems to occur below a threshold serum concentration of approximately 10-30 μg/mL. The half-life ranged from 19 days (linear portion of the profile) to 5 days (maximum clearance due to TMDD).

The PK of bimagrumab was not dose proportional over the range 0.1 to 30 mg/kg i.v. for AUClast, but did show dose-proportionality for Cmax. There was a slight accumulation of exposure (ratio of 1.25 based on AUCtau) following 3 consecutive monthly doses of 10 mg/kg i.v. Monthly administration of 3 mg/kg i.v. resulted in saturation of clearance for approximately one week (i.e. bimagrumab concentrations above the threshold), whereas 10 mg/kg provided saturation of clearance over the entire dosing interval of 4 weeks. The PK profile of healthy volunteers of Japanese descent, older adults up to 83 years of age, obese adults and patients with sIBM were similar to profiles of healthy younger adults.

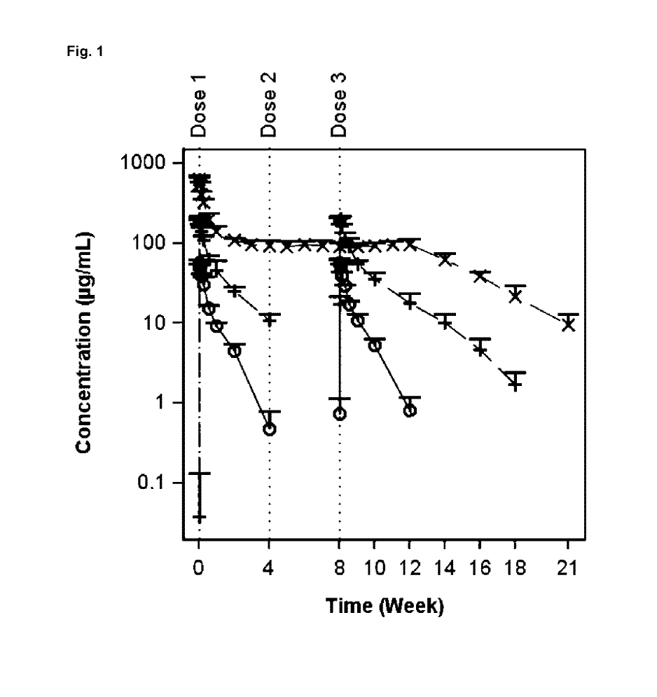

The PK profile was similar after a single i.v. dose of 30 mg/kg whether it was administered as a 30 minute or 2-hour infusion. PK profiles in sIBM and sarcopenia patients have been similar to the ones found in healthy subjects. The mean concentration profiles of the three cohorts from the multiple dose study (CBYM338X2102) are shown in FIG. 1.

### Human Pharmacodynamic Data

In the multiple dose study the primary PD endpoint of interest was the change in thigh muscle volume (TMV) from baseline to multiple distal time points. Mean TMV increased in all three cohorts that received bimagrumab and remained stable in subjects that received placebo. In Cohorts 1 (10 mg/kg) and 2 (3 mg/kg) measureable changes of 1.4% and 2.9% were observed after one week on drug and continued to increase in both cohorts stabilizing at approximately 5.7% in Cohort 1 and 4.9% in Cohort 2 across Weeks 8 (final dose) through 12. Cohort 3 showed a within group mean increase from baseline of 4.2% at Week 4 that stabilized at approximately 7% from Weeks 12 to 21. Subjects who received 3 or 10 mg/kg showed a range of improvement in TMV over placebo between 0.7% to 6.8% at Week 1 and 3.7% to 13% at Week 12. The range of improvement in Cohort 3 compared to placebo was 0 to 6% at Week 4 to 0 to 11% at Week 21 (EOS). TMV recovered toward baseline values by the end of study with Cohort 1 still 3.4% above baseline and Cohort 2 at baseline. All changes from baseline values were statistically different from placebo at all time points, except the final (end of study) measurement for 3 mg/kg.

Data from first interpretable results on 40 patients aged 65-86 years in the Proof of Concept trial in older sarcopenic adults with mobility limitations (CBYM338X2201) demonstrated:
Increases in TMV of approximately 8% (p<0.001) above baseline vs. placebo were observed at Weeks 8 and 16 and was maintained above baseline at Week 24.
A baseline by treatment interaction for gait speed as baseline mobility was related to treatment effect size. Stratifying the sample by gait speed performance of 0.8 m/s, a statistically and clinically meaningful treatment difference was observed in the subgroup of patients with slow gait speed (<0.8 m/s) at baseline (p=0.009).
Similarly 6MWT was influenced by baseline performance. A significant treatment effect (p=0.02) in patients with lower baseline 6MWT (<300 m) was seen at 16 weeks and maintained at 24 weeks/EOS.
Safety profile similar to that observed in healthy volunteers (aged 19-83 years) and patients with sporadic inclusion body myositis (up to age 78 years), with muscle spasms of mostly mild severity being the most frequent adverse event.

### Example 3

### A 28 Week, Randomized, Double-Blind, Placebo-Controlled, Multi-Center, Parallel Group Dose Range Finding Study to Assess the Effect of Monthly Doses of Bimagrumab 70, 210, and 700 Mg on Skeletal Muscle Strength and Function in Older Adults With Sarcopenia

Purpose and Rationale:

The purpose of this study is to determine the efficacy of repeat dosing with multiple dose levels of bimagrumab on patient function, skeletal muscle mass and strength in older adults with sarcopenia. In addition, this study will generate data on the safety, tolerability, and pharmacokinetics of bimagrumab in older adults with sarcopenia.

The randomized, parallel group, placebo-controlled design will allow an unbiased comparison between 3 different dose regimens of bimagrumab and placebo on changes in muscle quantity and patient physical function in a population of older adults with sarcopenia

Primary Objective(s):

The primary objective is to assess the effect of bimagrumab given intravenously every 4 weeks on the 6 minute walk distance test (6MWT) as assessed by change from baseline to week 25 relative to placebo in older adults with sarcopenia.

### Secondary Objectives:

To assess the effect of bimagrumab compared to placebo on the safety and tolerability of multiple doses of bimagrumab administered over 24 weeks as assessed by measures such as vital signs, clinical laboratory variables, electrocardiogram (ECG), echocardiogram, and adverse events (AE) in older adults with sarcopenia.

To assess the effect of bimagrumab compared to placebo on improvement in physical performance as measured by change from baseline to week 25 in the Short Physical Performance Battery (SPPB) score in older adults with sarcopenia.

To assess the effect of bimagrumab compared to placebo on improvement in mobility as measured by change from baseline at week 25 in gait speed (GS; measured as a component of the SPPB) over 4 meters in older adults with sarcopenia.

To assess the effect of bimagrumab on total lean body mass measured by DXA and appendicular skeletal muscle index (ASMI) as assessed by change from baseline to week 25 compared to placebo in older adults with sarcopenia.

### Study Design:

It is a randomized, double-blind, placebo-controlled, parallel group study design with approx. 280 sarcopenic patients randomized to four monthly treatments: placebo, bimagrumab 70 mg, bimagrumab 210 mg, or bimagrumab 700 mg.

The study will consist of a 20-day screening period followed by a 28-day run-in period, and a 24 week treatment period followed by a 4 weeks follow-up period. During the run-in period, all subjects will be introduced to a 3 times a week exercise program, daily vitamin D supplementation, and the performance measures. Towards the end of the run-in period, subjects will be re-assessed for eligibility (utilizing the baseline eligibility criteria) and qualified subjects will be randomly assigned to one of four monthly treatments.

### Population:

The study population will be community-dwelling men and women ages 70 years and older meeting the criteria for sarcopenia as defined by the European Working Group on Sarcopenia in Older People (EWGSOP) (Cruz-Jentoft et al 2010).

### Inclusion Criteria:

1. Men and postmenopausal women aged 70 years or older with self- reported mobility limitations such as difficulty standing up from a chair, walking for longer than 10 minutes on a flat surface or climbing a flight of stairs;
   Women are considered post-menopausal and not of child bearing potential if they have had 12 months of natural (spontaneous) amenorrhea with an appropriate clinical profile (e.g. age appropriate, history of vasomotor symptoms) or have had surgical bilateral oophorectomy (with or without hysterectomy) or tubal ligation at least six weeks ago. In the case of oophorectomy alone, only when the reproductive status of the woman has been confirmed by follow up hormone level assessment is she considered not of child bearing potential.
2. Gait speed over 4 meters of <0.8 m/s but ≧0.3 m/s at screening and baseline;
3. Appendicular skeletal muscle index (skeletal muscle in kg/height in m2) by DXA:
4. ≦7.26 kg/m2 for men and ≦5.5 kg/m2 for women to be assessed during screening.
5. JAPAN AND TAIWAN ONLY: ≦7.0 kg/m2 for men and ≦5.4 kg/m2 for women to be assessed during screening (Chen et al 2014);
6. Subjects must weigh at least 40.0 kg to participate in the study and have a body mass index (BMI) within the range of 18.0-30.0 kg/m2;
7. Usual dietary intake ≧20 kcal/kg body weight and ≧0.8 g protein/kg per day over the 4 weeks prior to screening estimated by an established method of diet assessment

### Exclusion Criteria:

Medical Conditions Limiting Performance of Physical Assessments
1. History of a lower limb fracture (e.g. femur, tibia) within the past 6 months with persistent negative impact on lower extremity function or any significant impairment or disease adversely impacting gait (e.g. intermittent claudication in advanced peripheral vascular disease, spinal stenosis, or severe osteoarthritis of the knee or hip);
2. Confirmed diagnosis of significant psychiatric disease (e.g. dementia/Alzheimer's disease, schizophrenia, depression or bipolar disorder);
3. Subjects with a Patient Health Questionnaire-9 (PHQ-9) score>10 at screening;
4. Neurological injury/disorder with significant persistent neurological or functional deficit (e.g. stroke with hemiparesis, spinal cord injury, muscular dystrophy, myopathy, myasthenia gravis, Parkinson's disease, peripheral polyneuropathy);
5. Ocular trauma, ophthalmologic surgery, or eye laser treatment within 6 months prior to screening;
6. Vitamin D deficiency defined as 25-OH-vitamin D levels<12.0 ng/mL at screening and baseline;
7. Hemoglobin concentration below 11.0 g/dL at screening.

Medical Conditions Associated With Muscle Loss
8. Chronic kidney disease [estimated glomerular filtration rate (GFR)<30 mL/min];
9. History of confirmed chronic obstructive pulmonary disease with a severity grade>2 on the Medical Research Council Dyspnea Scale;
10. Uncontrolled hypothyroidism or hyperthyroidism. Hypothyroid patients who have changed their dose of hormone replacement therapy in the 6 weeks prior to screening are not eligible for the study;
11. Underlying muscle diseases, including history of or currently active myopathy (e.g., dermatomyositis, polymyositis, etc) or muscular dystrophies;
12. Confirmed rheumatoid arthritis, acquired immunodeficiency syndrome (AIDS), or type 1 diabetes mellitus;
13. History of or ongoing gastrointestinal diseases known to cause malabsorption of protein or energy, such as inflammatory bowel disease, celiac disease, short bowel syndrome, pancreatic insufficiency;

Liver Related Conditions
14. Abnormal liver function tests such as SGOT (AST), SGPT (ALT), alkaline phosphatase, or serum bilirubin (except Gilbert's Disease). The investigator should be guided by the following criteria:
   Any single transaminase may not exceed 3×the upper limit of normal (ULN). A single parameter elevated up to and including 3×ULN should be re-checked as soon as possible, and always prior to enrollment/randomization, to rule out any lab error.
   If the total bilirubin concentration is increased above the ULN, total bilirubin should be differentiated into the direct and indirect reacting bilirubin. In any case, serum bilirubin should not exceed the value of 1.6 mg/dL (27 μmol/L).
15. Known history or presence of severe active acute or chronic liver disease (e.g., cirrhosis) or conditions with hepatotoxic potential (e.g. known gallbladder or bile duct disease, acute or chronic pancreatitis);

Cardiovascular Conditions
16. Systolic blood pressure>180 or <90 mm Hg or diastolic blood pressure>100 or <50 mm Hg at screening or baseline, or malignant hypertension.
17. Confirmed diagnosis of heart failure classified as New York Heart Association Class III and IV (e.g. cardiomyopathy), or hypertrophic cardiomyopathy;
18. History of unstable angina, myocardial infarction, coronary artery bypass graft surgery, or percutaneous coronary intervention (e.g. angioplasty or stent placement), deep vein thrombosis/pulmonary embolism within 6 months of screening or 1 year for drug-eluting stents;
19. Severe cardiac valve disorders or defects (e.g. aortic or mitral stenosis, or septal defects, or presence of artificial heart valve);
20. Severe pulmonary hypertension;
21. History of significant cardiac conduction/electrophysiological disorder, e.g. familial long QT syndrome or known family history of Torsades de Pointes or prolonged QT syndrome or QTcF≧450 msec (Fridericia Correction) for males and ≧460 msec for females at screening or baseline (by local ECG reading);
22. Confirmed significant cardiac arrhythmia (e.g. 2nd AV block Mobitz Type II/3rd degree heart block, SVT, VTach, automated cardioverter/defibrillator). Any current supra-ventricular arrhythmia with an uncontrolled ventricular response (mean heart rate>100 beats per minute [bpm]) at rest despite medical or device therapy;

Other Medical or Living Conditions
23. History of hypersensitivity to therapeutically administered antibodies.
24. Chest pain, severe shortness of breath, or occurrence of other safety concerns during the screening or baseline assessments.
25. Lack of peripheral venous access
26. Active cancer (i.e., under current treatment), or cancer requiring treatment in the last 5 years excluding nonmelanoma skin cancers or cancers with excellent prognosis (e.g., early stage prostate or breast cancer, carcinoma in situ of the uterine cervix);
27. Uncontrolled type 2 diabetes mellitus (i.e. HbA1C≧8.0% or frequent hypoglycemia);
28. Significant coagulopathy, platelet count less than 75,000/mm3;
29. Active systemic infection requiring hospitalization or treatment with IV anti-infectives or antibiotics within 4 weeks of screening;
30. Any chronic active infection (e.g., HIV, hepatitis B or C, tuberculosis, etc).

Subjects receiving chemoprophylaxis for latent tuberculosis infection are eligible for the study;
31. Active alcohol/drug abuse, or alcohol/drug treatment<12 months prior to screening; subjects having successfully completed an alcohol/drug treatment program>12 months prior to screening with sustained abstinence are eligible’;
32. Subject has any medical condition or laboratory finding during screening (e.g. an unexplained or clinically significant lab result), which, in the opinion of the investigator may interfere with participation in the study, might confound the results of the study, or pose an additional safety risk in administering bimagrumab;
33. Subject plans to move out of the study area within 12 months or be out of study area for >4 weeks continuously;
34. Individuals who require routine and regular (e.g., daily) assistance from another person to complete one or more activities of daily living (ADL; e.g. bathing, dressing, toileting) regardless of where they reside;
35. Men with hypogonadism (testosterone<250 ng/dl) at screening;
36. Subjects with a Mini Mental State Examination score<24 at screening;

Prohibited Medication
37. Use of any therapies known to affect muscle mass, including androgens, androgen supplements [including over-the-counter dehydroepiandrosterone (DHEA)], gonadotropin releasing hormone (GnRH) analogues, anti-androgens, anti-estrogens (e.g. tamoxifen), progestins with known androgenic component (e.g. norethindrone acetate, megestrol acetate, high-dose tibolone (2.5 mg), recombinant human growth hormone, growth hormone receptor antagonists (e.g., pregvisomant), oral selective beta-2 agonists, or dronabinol within 3 months prior to randomization; and any nutritional supplement other than protein marketed as a muscle anabolic.
38. Currently enrolled in, or discontinued within the last 30 days (or 5 half-lives of enrollment or until PD effect is expected to return to baseline, whichever is longer or longer if required by local regulations) from a clinical trial involving an investigational drug or off-label use of a drug, or are concurrently enrolled in any other type of medical research judged to be scientifically or medically incompatible with this study.
39. Ongoing corticosteroid use or history of systemic corticosteroid use for at least 3 months (in the last year) prior to screening or randomization at a daily dose greater than or equal to 10 milligram (mg) prednisone equivalent;
40. Use of vascular endothelial growth factor (VEGF) inhibitors (e.g. bevacizumab) within 6 months prior to randomization;
41. Use of antibody immunosuppressive therapy (e.g., rituximab or iv immunoglobulin, TNFalpha inhibitors) within 6 months of randomization;
42. Use of non-antibody immunosuppressive therapy (e.g. cyclosporine, methotrexate, tacrolimus, cyclophosphamide) within 3 months of randomization
43. Chronic use of a beta blocker initiated within the three months prior to screening.

Investigational and Reference Therapy:

Placebo, bimagrumab 70 mg, bimagrumab 210 mg, or bimagrumab 700 mg

Efficacy Assessments:
6 minute walking test (6MWT) to assess functional improvement
Hand grip strength test to assess strength
Short physical performance battery (SPPB) to assess functional improvement
Gait Speed (GS is a component of SPPB) to assess functional improvement
400 m walk test to assess functional improvement
Total lean body mass (LBM) and appendicular skeletal mass index (ASMI) assessed by DXA to measure lean body mass and skeletal muscle mass of the arms and legs, respectively.
Patient reported Outcome questionnaires

Safety Assessments:
Physical examination
Vital signs
Height and weight
Laboratory evaluations
Urinalysis
ECG
Echocardiography
Falls diary
PK
IG

Other Assessments:
Tri-axial accelerometer fitness monitoring
PG

Data Analysis:

The primary variable (6MWT) measured at 6 months in the core study phase will be analyzed by the MCP-MOD methodology, Pinheiro et al. (2006). A set of three candidate scale-location families will be specified, and optimal contrasts will be derived from these families.

### Rationale for Study Design

The randomized, parallel group, placebo-controlled design will allow an unbiased comparison between 3 different dose regimens of bimagrumab and placebo on changes in muscle quantity and patient physical function in a population of older adults with sarcopenia.

The study population will be comprised of men and women aged 70 years or older with characteristics of sarcopenia and muscle-associated slow gait speed (GS). The population enrolled in this study should reflect the general heterogeneity of elderly people with low skeletal muscle mass and mobility limitation with regard to co-morbidities, polypharmacy, physical functional status, physiological reserve, and physical activity patterns. Data on drug safety, tolerability, pharmacokinetics and pharmacodynamics from this design and population, should provide an assessment of possible beneficial or adverse effects of bimagrumab in the larger population of elderly adults with similar co-morbidities, functional status and mobility limitations.

Approximately 280 patients will be randomized in a 1:1:1:1 ratio (0 mg:70 mg:210 mg:700 mg) for approximately 70 patients per treatment arm with 60 per arm expected to complete. Randomization will be used to account for the expected heterogeneity of the geriatric sample population and to minimize selection, age, gender and baseline differences between groups. It is expected that patients administered bimagrumab will demonstrate a greater increase in muscle mass (ASMI) after receiving the drug compared to patients receiving placebo and that this increase in muscle will translate into an improvement in physical function seen as an increase in the distance walked in six minutes (6MWT), improvement in the Short Physical Performance Battery (SPPB) score and other secondary outcomes.

After completing 4 weeks of performing the exercise program patients are expected to improve slightly on the performance measures. Therefore, baseline assessments that will be used for determining change over time in the identified outcomes will be taken at the end of the 4-week run-in period. To standardize the exercise program across all patients, the schedule of three exercise sessions per week will be maintained throughout the study.

The primary endpoint of this study will be distance completed during the 6MWT following 24-weeks of study drug. We hypothesize that a clinically meaningful change (>50 m) in the 6MWT distance will be observed by Week 25, one month after the last dose. Based on preliminary results, positive effects on the 6MWT distance may be observed earlier. The SPPB (standing balance, 4-meter gait speed (GS) and repetitive chair rise), the 400 m walk test, hand grip strength, and patient reported outcomes of health status and function (GPAQ, SF36, EQ-5D) will provide data to assess the potentially broader clinical impact of a change in muscle quantity on improvements in patient functional status (see Section 6.5).

A novel mobility monitoring technology may be used to track daily physical activity and falls. This exploratory outcome measure will be used to validate the ability of this fitness monitor to record falls and voluntary physical activity in this patient population (see Section 6.9.1).

Biomarker samples have been incorporated into the trial to further explore the identification of valid and reliable biomarkers of clinical benefit with bimagrumab to predict changes in total lean body mass after multiple dose treatments combined with regular exercise and ideally to predict functional improvement (see Section 6.5 and Section 6.9).

### Rationale for Dose/Regimen, Duration of Treatment

Dose and Frequency

This study will evaluate fixed i.v. doses of bimagrumab 70, 210, or 700 mg administered every 4 weeks over a 21-week period for a total of six doses.

The fixed dose equivalent of the mg/kg doses used in previous studies was calculated based on the mean patients' weight in the recent bimagrumab sarcopenia PoC study CBYM338X2201 (70 kg). Six doses will be used to evaluate the durability of treatment on the expected improvement in physical function and the time course for the range of performance assessments and to avoid being misled by early changes that are not maintained with continued dosing (Papanicolaou et al 2013).

One and two i.v. doses of 30 mg/kg of bimagrumab have been evaluated in multiple studies and shown to be safe and well tolerated in healthy men and women up to 78 years of age as well as patients with sporadic inclusion body myositis (sIBM) or sarcopenia up to 86 years of age. Safety data from the multiple dose study (CBYM338X2102) showed an improved safety/tolerability profile with three doses of either 3 or 10 mg/kg compared to a single dose of 30 mg/kg.

Six monthly doses of 700 mg (10 mg/kg equivalent) of bimagrumab are expected to sufficiently block the ActRII receptors enabling an efficacious response for a total of approximately 7 months (treatment period) based on data from earlier clinical studies (see FIG. 1-2). The actual duration of receptor blockade on skeletal muscle with specific dose levels has not been determined. Bimagrumab is not expected to adversely interact with other drugs used by individuals in this study based on antibody biology and experience with bimagrumab in older patient populations, including sarcopenia with mobility limitation.

In healthy volunteers (CBYM338X2101), thigh muscle volume (TMV) assessed by MRI increased comparably for single doses of 10 mg/kg and 30 mg/kg, although the effect of the 30 mg/kg dose lasted longer. With three sequential monthly doses of bimagrumab (CBYM338X2102), there was a comparable increase in TMV in healthy adults at 3 mg/kg and 10 mg/kg, although it is believed that the 3 mg/kg dose causes complete receptor occupancy for roughly one week whereas it is around four weeks with the 10 mg/kg dose (see Section 1.1.3). Thus, both the 3 mg/kg dose equivalent (210 mg) and the 10 mg/kg dose equivalent (700 mg) are expected to be efficacious in the proposed study with sarcopenia patients, with fewer side effects than 30 mg/kg. In healthy volunteers (CBYM338X2101), a limited and transient effect on the TMV was observed after infusion of a single dose of 1 mg/kg bimagrumab. The 1 mg/kg dose is therefore expected to be a non-effective or a minimally effective dose in this study.

Because little accumulation was reported after 3 consecutive monthly doses (˜factor of 1.25 based on AUCtau comparison) and because steady state was reached after 3 doses, exposure to bimagrumab after 6 monthly doses is expected to be comparable to that observed after the three doses, which has been shown to be safe and well tolerated. The treatment duration of a total of 6 months is supported by the 26-week toxicology studies in Cynomolgus monkeys as shown in the following table.

### Comparative Pharmacokinetics in Cynomolgus Monkey and Humans:

| | | | Dose | | |
|---|---|---|---|---|---|
| | | | (mg/kg/week | AUC | Cmax |
| Species | Gender | Route | or mg/kg)^{a} | (μg*d/mL)^{b} | (μg/mL) |
| Cynomolgus | M (N = 6) | i.v. | 300 | 35600 | 11700 |
| | F (N = 6) | i.v. | 300 | 30500 | 10800 |
| Human | M + F^{c} | i.v. | 3 | 261 | 56.3 |
| | (N = 11) | | 10 | 1100 | 195 |

^{a})Multiple dose for cynomolgus monkey (26 weeks toxicology study, weekly administration) and for Human (CBYM338X2102 monthly administration)
^{b})AUC0-168h (i.e. AUCtau) for cynomolgus monkey after the last dose of the 26 weeks toxicology study at the NOAEL (Day 176) and AUC0-28 d (i.e. AUCtau) after the last dose for Human in the CBYM338X2102 study.
^{c})Male and Female pooled for mean as there were too few females to derive summary statistics

### Rationale for Choice of Comparator:

A placebo infusion will be used as the comparator in this placebo-controlled study; no drug comparator will be used. Placebo is used because several of the outcome measures are behavioral in nature and dependent on a patient's or observer's beliefs. Therefore, knowing treatment assignment may bias the important outcome measures. In addition, placebo-controlled studies provide the optimal situation to distinguish adverse events caused by a drug from those resulting from underlying conditions or “background noise”. As there is no approved pharmacotherapy for sarcopenia and it is not known if bimagrumab may be efficacious, the use of placebo is also ethically appropriate.

### Efficacy/Pharmacodynamic Assessments

Pharmacodynamic assessments are detailed below. Efficacy measurements in the study will include:
6 minute walk test (6MWT) to assess physical function
Hand grip strength test to assess strength
Short physical performance battery (SPPB) to assess physical function
Gait Speed (GS is a component of SPPB) to assess physical function
400 m walk test to assess physical function
Total lean body mass (LBM) and appendicular skeletal mass index (ASMI) assessed by DXA to measure lean body mass and skeletal muscle mass of the arms and legs, respectively.

Physical function assessments will be done at all sites by trained site personnel to ensure standard results.

6 Minute Walk Test:

The 6 minute walk test (6MWT) is a simple, economical and reproducible test that measures how many meters a person can walk in 6 minutes. Repeated measurement of the 6MWT over time has been used in studying numerous musculoskeletal, pulmonary, and cardiovascular conditions and is a validated outcome in investigational drug trials.

Patients will be instructed by the test administrator using a script and established testing protocol. The testing should be conducted on an individual basis (patient and testers) with no additional audience or support other than that of the trained personnel conducting the test. If a walking aid is required at baseline, patients will be asked to use the least assistive walking aid that in their opinion will enable them to complete the 6MWT test safely. Patients should be encouraged to use the same walking aid when performing all tests throughout the study. A change in walking aid to perform the test is permitted if required for safety reasons (e.g. deterioration of balance). The testing should occur at approximately the same time of the day as the baseline assessment to prevent any possible diurnal variations. The same test administrator should perform all repeat tests on a patient whenever possible to reduce technician-related differences in test performance.

Hand Grip Strength:

Handgrip dynamometry is frequently used in clinical and research settings as a proxy to assess overall muscle strength. The Jamar® Hydrolic Hand Dynamometer is a fast, reliable and easy to use device commonly used by rehabilitation specialists in different patient populations, including geriatric patients.

Short Physical Performance Battery:

The Short Physical Performance Battery (SPPB) has been shown to be highly predictive of subsequent disability, hospitalization, institutionalization, and mortality in community-dwelling elders in epidemiological studies and outpatient clinics (Guralnik et al 2000; Studenski at al 2003). The disability remains even after adjustment for level and severity of comorbidity and self-report functional status. Collectively, SPPB might be considered to be a nonspecific but highly sensitive indicator of global health status reflecting several underlying physiological impairments.

The SPPB evaluates lower extremity function by measuring three domains of physical function: maintenance of standing balance, usual gait speed and lower extremity strength and power. The corresponding tasks include three static positions with decreasing base of support to challenge balance, walking at usual speed over 4-meters and, the ability to rise from a chair without the use of the arms once and then five times consecutively. The final score is a composite of the three groups of tasks and uses a standardized scale of 0-12, with the higher score reflecting a higher level of function. A change of 1.0 on the SPPB score is considered clinically relevant.

Gait Speed:

Gait speed in this study will be assessed as part of the SPPB, over a 4 meter distance of a 6 meter course. This test assesses a person's usual walking speed, which is defined as the speed a person normally walks from one place to another (e.g., walking from one store to another).

Usual gait speed represents one of the most suitable physical performance measures to evaluate older persons. Gait speed is associated with physical activity levels, changes in strength of lower extremity muscles, frailty and falls (Newman et al 2003, Chandler et al 1998, Cesari et al 2005).

Gait speed is a well-established measure of physical function, it has shown to predict future disability in diverse community-dwelling elderly populations and is sensitive to changes in physical status in response to an intervention (e.g. physical activity and rehabilitation) (Barthuly et al 2012). Poor functional performance as measured by slow or declining gait speed is related to an increased risk of disability, hospitalization and mortality (Studenski et al 2011), whereas improvements in gait speed are related to reductions in mortality risk (Hardy et al 2007). For these reasons, gait speed has been suggested as a key indicator of overall health in the geriatric population.

400 Meter Walk Test:

The 400 meter walk test is a measure of cardiorespiratory fitness, lower extremity muscle function and general mobility. During this self-paced walking test, patients are instructed to walk 400 meter at their usual pace or without any expectation of time. The ability to walk 400 meters in less than 15 minutes has been suggested as an indicator of sufficient capacity for community ambulation. ‘Mobility disability’ has been defined as the inability to walk 400 meters in 15 minutes or less. A healthy older adult should be able to complete the 400 meter test in 6 minutes (Simonsick et al 2000). The 400 meter distance is also comparable to the reference distance (¼ mile) of a commonly performed self-report measure of mobility- related difficulty (Rosow and Breslau 1966). The 400 meter walk is the final performance assessment administered at each testing time point; adequate rest (a minimum of 60 minutes) will be provided between the 6MWT and the 400 meter walk assessment. Alternatively, the 400 meter walk test can be administered on a separate day.

Total Lean Body Mass and Appendicular Skeletal Mass Index (ASMI) Assessed by DXA:

Dual energy X-ray absorptiometry (DXA) will be used to assess changes in total lean body mass (LBM) and appendicular skeletal mass index (ASMI). DXA instruments use an x-ray source that generates and is split into two energies to measure bone mineral mass and soft tissue from which fat and fat-free mass (or lean body mass) are estimated. The exam is quick (˜5-6 min), precise (0.5-1%) and non-invasive. DXA scanners have the precision required to detect changes in muscle mass as small as 5%.

Radiation exposure from DXA scans is minimal. The National Council of Radiation Protection and Measurements (NCRP) has recommended the annual effective dose limit for infrequent exposure of the general population is 5,000 μSv and that an annual effective dose of 10 μSv be considered a Negligible Individual Dose. The effective dose of a DXA whole body scan on an adult is 5 μSv. The total amount of radiation exposure per subject from DXA in this study will be about 25 μSv. This amount of radiation is equivalent to approximately 3.6 days of background exposure (approx. 0.3 μSv per hour at sea level).

Studies have shown that quality assurance is an important issue in the use of DXA scans to determine body composition. DXA instrument manufacturer and model should remain consistent and their calibration should be monitored throughout the study. Use of a standardized scan acquisition protocol and appropriate and unchanging scan acquisition and analysis software is essential to achieve consistent results. Likewise, because of variability in interpretation of the scans, it is important to utilize centralized scan analysis by experienced staff.

### REFERENCES

The entire content of the following references, in particularly their definitions and descriptions in relation to sarcopenia, muscle mass, muscle strength, (physical) performance, mobility, weakness, of clinical test methods and of diagnosis criteria, are incorporated herein by reference.

Barthuly A M, Bohannon R W, Gorack W (2012) Gait speed is a responsive measure of physical performance for patients undergoing short-term rehabilitation. Gait Posture; 36(1):61-4.

Baumgartner R N, Koehler K M, Gallagher D, et al (1998) Epidemiology of sarcopenia among the elderly in New Mexico. Am J Epidemiol 1998; 147: 755-63.

Biomarkers Definitions Working Group. (2001) Biomarkers and surrogate endpoints: Preferred definitions and conceptual framework. Clinical Pharmacology & Therapeutics; 69:89-95.

Cesari M, Kritchevsky S B, Penninx B W, et al (2005) Prognostic value of usual gait speed in well-functioning older people-results from the Health, Aging and Body Composition Study. J Am Geriatr Soc; 53:1675-80.

Chandler J M, Duncan P W, Kochersberger G, et al (1998) Is lower extremity strength gain associated with improvement in physical performance and disability in frail, community-dwelling elders? Arch Phys Med Rehabil; 79(1):24-30.

Chen J L, Walton K L, Winbanks C E, Murphy K T, Thomson R E, Makanji Y, Qian H, Lynch G S, Harrison C A, Gregorevic P (2014). Elevated expression of activins promotes muscle wasting and cachexia. FASEB J. April; 28(4):1711-23.

Chen L-K, Liu L-K, Woo J, et al (2014) Sarcopenia in Asia: Consensus report of the Asian working group for sarcopenia. JAMDA; 15 95-101.

Cruz-Jentoft A J, Baeyens J P, Bauer J M, et al (2010) Sarcopenia: European consensus on definition and diagnosis Report of the European Working Group on Sarcopenia in Older People. Age and Ageing; 39: 412-423.

Dam T-T, Peters K W, Fragala M, et al. (2014) An evidence-based comparison of operational criteria for the presence of sarcopenia. J Gerontol Med Sci 69: 584-590.

Fielding R, Vellas B, Evans W, et al (2011) Sarcopenia: An Undiagnosed Condition in Older Adults. Current Consensus Definition: Prevalence, Etiology, and Consequences. International Working Group on Sarcopenia. J Am Med Dir Assoc; 12: 249-256.

Gottdiener J S, Bednarz J, Devereux R, et al (2004) American Society of Echocardiography Recommendations for Use of Echocardiography in Clinical Trials. J Am Soc Echocardiogr; 17:1086-1119.

Guralnik J M, Ferrucci L, Pieper C F, et al (2000) Lower extremity function and subsequent disability: consistency across studies, predictive models, and value of gait speed alone compared with the short physical performance battery. J Gerontol A Biol Sci Med Sci; 55(4):M221-31.

Hardy S E, Perera S, Roumani Y F, et al (2007) Improvement in usual gait speed predicts better survival in older adults. J Am Geriatr Soc; 55(11):1727-34.

Janssen I, Shepard D S, Katzmarzyk P T, et al (2004) The healthcare costs of sarcopenia in the United States. J Am Geriatr Soc. January; 52(1):80-5.

Kwon S, Perera S, Pahor M, et al (2009) What is a meaningful change in physical performance? Findings from a clinical trial in older adults (the LIFE-P study). J Nutr Health Aging. 2009 June; 13(6):538-44.

Lee S J, Lee Y S, Zimmers T A, et al (2010) Regulation of muscle mass by follistatin and activins. Mol. Endocrinol.; 24 (10):1998-2008.

Lee S J, Reed L A, Davies M V, et al (2005) Regulation of muscle growth by multiple ligands signaling through activin type II receptors. Proc. Natl. Acad. Sci. U.S.A; 102 (50):18117-18122.

Morley J E, Abbatecola A M, Argiles J M, et al (2011) Sarcopenia with limited mobility: An international consensus. J Am Med Dir Assoc 12: 403-409.

Muscaritoli M, Anker S D, Argiles J, et al (2010) Consensus definition of sarcopenia, cachexia and pre-cachexia: joint document elaborated by Special Interest Groups (SIG) “cachexia-anorexia in chronic wasting diseases” and “nutrition in geriatrics”. Clin Nutr. April; 29(2):154-9. doi: 10.1016/j.clnu.2009.12.004.

Newman A B, Haggerty C L, Kritchevsky S B, et al; Health ABC Collaborative Research Group (2003). Walking performance and cardiovascular response: associations with age and morbidity—the Health, Aging and Body Composition Study. J Gerontol A Biol Sci Med Sci; August; 58(8):715-20.

Papanicolaou D A, Ather S N, Zhu H, et al (2013) A phase IIA randomized, placebo-controlled clinical trial to study the efficacy and safety of the selective androgen receptor modulator (SARM), MK-0773 in female participants with sarcopenia. J Nutr Health Aging. 2013; 17(6):533-43

Perera, S, Moody S H, Woodman, R C, et al (2006) Meaningful change and responsiveness in common physical performance measures in older adults. J Am Geriatr Soc. May; 54; (5):743-9

Pinheiro J C, Bornkamp B. and Bretz F. (2006). Design and analysis of dose finding studies combining multiple comparisons and modeling procedures, Journal of Biopharmaceutical Statistics, 16, 639-656

Rosow I, Breslau N (1966) A Guttman health scale for the aged. J Gerontol. October; 21(4):556-9.

Simonsick E M, Gardner A W, and Poehlman E T (2000) Assessment of physical function and exercise tolerance in older adults: reproducibility and comparability of five measures. Aging (Milano). August; 12(4):274-80

Stabin M G (2009); Nuclear Medicine Radiation Risks. J Nucl Med. February 17.

Studenski S A, Peters K W, Alley D E, et al (2014) The FNIH Sarcopenia Project: Rationale, study description, conference recommendations and final estimates. J Gerontol A Biol Sci Med Sci 69(5): 547-558.

Studenski S, Perera S, Patel K, et al (2011) Gait speed and survival in older adults. JAMA; 305(1):50-8.

Studenski S, Perera S, Wallace D, et al (2003) Physical performance measures in the clinical setting. J Am Geriatr Soc; 51(3):314-22.

Tomlinson B, Cruickshank J M, Hayes Y, et al (1990) Selective beta-adrenocceptor partial agonist effects of pindolol and xamoterol on skeletal muscle assessed by plasma creatinine kinase changes in healthy subjects. Brj J Pharmac; 30: 665-672.

Trendelenburg A U, Meyer A, Jacobi C, et al (2012) TAK −1/p38/nNFkB signaling inhibits myoblast differentiation by increasing levels of Activin A. Skeletal Muscle; 2(1):3.

### ABBREVIATIONS/DEFINITIONS

6MWT 6 minute walk test
abs absolute
ADA Anti-drug antibody
AE adverse event
AF Atrial fibrillation
ALT alanine aminotransferase
ALP alkaline phosphatase
ANCOVA analysis of covariance
ActRIIA/B type II activin receptors A and B
AST aspartate aminotransferase
AWGS Asian Working Group for Sarcopenia b.i.d. twice a day
BMI Body Mass Index
BUN blood urea nitrogen
CD-ROM compact disc-read only memory
CFR Code of Federal Regulation
CK creatinine kinase
CRF Case Report/Record Form (paper or electronic) CO2 carbon dioxide
CRO Contract Research Organization
C-SSRS Columbia Suicide Severity Rating Scale
CTC Common Toxicity Criteria
CV coefficient of variation
EC Ethics committee
ECG Electrocardiogram
EDC Electronic Data Capture
ELISA Enzyme-linked immunosorbent assay
EWGSOP European Working Group on Sarcopenia in Older People
DMC Data Monitoring Committee
FDA Food and Drug Administration
FSH follicle stimulating hormone
GCP Good Clinical Practice
GDF-11 Growth differentiation factor 11
γ-GT Gamma-glutamyl transferase GLP Good laboratory practice
GS Gait speed
h hour
HIV human immunodeficiency virus
ICH International Conference on Harmonization of Technical Requirements for Registration of Pharmaceuticals for Human Use
IEC Independent Ethics Committee i.v. intravenous
IRB Institutional Review Board
IRT Interactive Response Technology
LFT Liver function test (raised serum transaminases and/or bilirubin levels)
LDH lactate dehydrogenase
LIVI liquid in vial
LLQ lower limit of quantification
LLN lower limit of normal
MedDRA Medical dictionary for regulatory activities
mg Milligram(s)
MI Myocardial infarction
ml milliliter(s)
MMSE Mini Mental State Examination
OC/RDC Oracle Clinical/Remote Data Capture
o.d. once a day
PA posteroanterior
PD pharmacodynamic(s)
PIQ Protein Intake Questionnaire
PK pharmacokinetic(s)
p.o. oral(ly)
PRO Patient Reported Outcome
RBC red blood cell(s)
REB Research Ethics Board
SAE serious adverse event
SCID Severe Combined Immunodeficiency
SD standard deviation
SGOT serum glutamic oxaloacetic transaminase
SGPT serum glutamic pyruvic transaminase
sIBM Sporadic Inclusion Body Myositis
SPPB Short Physical Performance Battery
SUSAR Suspected Unexpected Serious Adverse Reactions
TBL total bilirubin
TGF-β transforming growth factor beta
TK toxicokinetics
TMDD target mediated drug disposition
TMV thigh muscle volume
ULN upper limit of normal
ULQ upper limit of quantification
WBC white blood cell(s)
WHO World Health Organization
WOCBP women of childbearing potential
Cmax: The observed maximum plasma (or serum or blood) concentration following drug administration [mass/volume]
Cmin: The lowest observed plasma (or serum or blood) concentration following drug administration [mass/volume]
Tmax: The time to reach the maximum concentration after drug administration [time]

1. A method of treating a patient suffering from age-related sarcopenia, comprising administering a myostatin or activin antagonist to a patient in need thereof.

2. The method according to claim 1, wherein the patient is a male or a menopausal female aged 50 years or older.

3. The method according to claim 1, wherein the myostatin or activin antagonist is an anti-ActRII receptor antibody.

4. The method according to claim 3, wherein the anti-ActRII receptor antibody is bimagrumab.

5. The method according to claim 4, wherein the myostatin or activin antagonist is administered intravenously at a dose of about 70 mg once every 4 weeks.

6. The method according to claim 4, wherein the myostatin or activin antagonist is administered intravenously at a dose of about 210 mg once every 4 weeks.

7. The method according to claim 4, wherein the myostatin or activin antagonist is administered intravenously at a dose of about 700 mg once every 4 weeks.

8. The method according to claim 1, wherein the treatment comprises an increase in skeletal muscle mass indicated by an increase of AL(B)M adjusted for body mass index (BMI) to reach latest after 24 weeks under treatment a value of at least 0.789 kg for a male or at least 0.512 kg for a female, said AL(b)M being measured by dual energy X-ray absorptiometry (DXA), and an increase in muscle strength indicated by reaching a value of at least 26 kg for a male or 16 kg for a female in the handgrip strength test latest after 24 weeks under treatment.

9. The method according to claim 1, wherein the treatment comprises an increase in skeletal muscle mass indicated by an increase of appendicular skeletal muscle index (ASMI) to reach latest after 24 weeks under treatment a value of at least 7.26 kg/m² for a male or at least 5.5 kg/m² for a female, said ASMI being defined as appendicular skeletal muscle mass divided by the square of height ASMI and being measured by dual energy X-ray absorptiometry (DXA), and an increase in muscle strength indicated by reaching a value of at least 30 kg for a male or 20 kg for a female in the handgrip strength test latest after 24 weeks under treatment.

10. The method according to claim 1, wherein the treatment comprises an increase in physical performance (or mobility increase) indicated by an increase of gait speed over a 4-m course (4MGS) by at least 0.05 m/s compared to the data before treatment (baseline) and an increase in (skeletal) muscle mass indicated by an increase of appendicular skeletal muscle index (ASMI) to reach latest after 24 weeks under treatment a value of at least 7.26 kg/m² for a male or at least 5.5 kg/m² for a female, said ASMI being defined as appendicular skeletal muscle mass divided by the square of height and being measured by dual energy X-ray absorptiometry (DXA).

11. The method according to claim 1, wherein sarcopenia is defined by the criteria of low muscle mass as indicated by an AL(B)M adjusted for body mass index (BMI) of ≦0.789 kg for a male or ≦0.512 kg for a female, said AL(b)M being measured by dual energy X-ray absorptiometry (DXA) and by the criteria of low muscle strength as indicated by a value of <26 kg for a male or <16 kg for a female in the handgrip strength test.

12. The method according to claim 1, wherein sarcopenia is defined by the criteria of low muscle mass as indicated by an appendicular skeletal muscle index (ASMI) of ≦7.26 kg/m² for a male or ≦5.5 kg/m² for a female, said ASMI being defined as appendicular skeletal muscle mass divided by the square of height, said ASMI being measured by dual energy X-ray absorptiometry (DXA) and by the criteria of low muscle strength as indicated by a value of <30 kg for a male or <20 kg for a female in the handgrip strength test.

13. The method according to claim 1, wherein sarcopenia is defined by the criteria of low physical performance (or mobility limitations) indicated by a gait speed over a 4-m course of ≦1 m/s, preferably ≦0.8 m/s, or more preferably <0.8 m/s and by the criteria of low muscle mass as indicated by an appendicular skeletal muscle index (ASMI) of ≦7.26 kg/m² for a male or ≦5.5 kg/m² for a female, said ASMI being defined as appendicular skeletal muscle mass divided by the square of height, said AL(B)M being measured by dual energy X-ray absorptiometry (DXA).

14. The method according to claim 4, wherein prior to said administering step bimagrumab is provided as a concentrated aqueous solution at a concentration from 100 to 200 mg/mL.

15. The method according to claim 14, wherein said concentrated aqueous solution is diluted for intravenous administration with an isotonic aqueous solution to form a diluted solution, and wherein the concentration of bimagrumab in the diluted solution is from 0.2 to 10 mg/mL.

16. The method according to claim 15, wherein said diluted solution is intraveneously administered to the patient with an infusion flow rate of 1-10 mL/min.


SEQUENCE LISTING (9 sequences)

SEQ ID NO: 1 - protein, 10 aa, Artificial
  Heavy chain CDR1
GYTFTSSYIN

SEQ ID NO: 2 - protein, 17 aa, Artificial
  Heavy chain CDR2
TINPVSGSTSYAQKFQG

SEQ ID NO: 3 - protein, 6 aa, Artificial
  Heavy chain CDR3
GGWFDY

SEQ ID NO: 4 - protein, 14 aa, Artificial
  Light chain CDR1
TGTSSDVGSYNYVN

SEQ ID NO: 5 - protein, 11 aa, Artificial
  Light chain CDR2
LMIYGVSKRPS

SEQ ID NO: 6 - protein, 10 aa, Artificial
  Light chain CDR3
GTFAGGSYYG

SEQ ID NO: 7 - protein, 217 aa, Artificial
  light chain
QSALTQPASVSGSPGQSITISCTGTSSDVGSYNYVNWYQQHPGKAPKLMIYGVSKRPSGVSNRFSGSKSGNTASLTISGLQAEDEADYYCGTFAGGSYYGVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 8 - protein, 217 aa, Artificial; identical to SEQ ID NO: 7
  light chain

SEQ ID NO: 9 - protein, 445 aa, Artificial
  heavy chain
QVQLVQSGAEVKKPGASVKVSCKASGYTFTSSYINWVRQAPGQGLEWMGTINPVSGSTSYAQKFQGRVTMTRDTSISTAYMELSRLRSDDTAVYYCARGGWFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK